(19) United States
(12) Patent Application Publication
(10) Pub. No.: US 2016/0253464 A1
(43) Pub. Date: Sep. 1, 2016

(54) APPOINTMENT SCHEDULING AND CHECK IN

(71) Applicant: Theranos, Inc., Palo Alto, CA (US)

(72) Inventors: Sunny Balwani, Palo Alto, CA (US); Elizabeth A. Holmes, Palo Alto, CA (US)

(21) Appl. No.: 15/054,047

(22) Filed: Feb. 25, 2016

Related U.S. Application Data
(63) Continuation of application No. PCT/US2014/054572, filed on Sep. 8, 2014.
(60) Provisional application No. 61/875,108, filed on Sep. 8, 2013.
(60) Provisional application No. 61/899,869, filed on Nov. 4, 2013.
(60) Provisional application No. 61/900,985, filed on Nov. 6, 2013.
(60) Provisional application No. 62/001,542, filed on May 21, 2014.

(51) Int. Cl. G06F 19/00; G06Q 10/02; G06Q 10/10

(52) U.S. Cl. CPC G06F 19/327; G06Q 10/1095; G06Q 10/02

(57) ABSTRACT

Systems and methods for appointment scheduling and check in are provided. The systems and methods facilitate appointment check at various locations including health service centers, and improve accuracy and efficiency of appointment scheduling.

### BACKGROUND

Facilities which offer on-site services to customers may face two objectives which are at least partially in tension with each other: 1) the objective to have sufficient resources (e.g. personnel, materials) on hand so as to efficiently meet the needs of customers who arrive at the facility; and 2) the objective to not have excess resources on hand such that resources are wasted.

In addition, facilities which offer services to customers on an appointment basis may encounter inefficiencies when a customer fails to arrive for his or her appointment at a scheduled time. The customer may, for example, arrive late and interfere with later-scheduled appointments for other patients, or the customer may, for example, not arrive at all, resulting in wasted resources by the facility.

Once a customer arrives at a facility for a service, a customer may encounter difficulties in checking in for the appointment. For example, there may be a long line at a check-in site. The long line may compound a situation where, for example, a customer arrives at the facility late for an appointment, and then needs to wait excessively long in order to check in for the appointment for which he or she is already late.

Accordingly, improvements in systems and methods for scheduling and check-in for appointments are needed.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Provided herein are embodiments for appointment scheduling and check in.

In embodiments, provided herein is a computer-implemented method for evaluating the risk of a patient's tardy arrival for a scheduled appointment at a time at a health service center, the method comprising: obtaining at least a first tardiness risk factor and a second tardiness risk factor for the patient for the scheduled appointment, obtaining, from a computer memory device and with the aid of a processor, a first quantified tardiness risk value for the first tardiness risk factor and a second quantified tardiness risk value for the second tardiness risk factor; and combining, according to an algorithm and with the aid of a processor, the first quantified tardiness risk value and the second quantified tardiness risk value, to generate a total tardiness risk value. Optionally, the method may further comprise comparing the total tardiness risk value to a threshold tardiness risk value, and, if the total tardiness risk value exceeds the threshold tardiness risk value, taking an action. Optionally, the method may further comprise taking an action based on the value of the total tardiness risk value.

Optionally, in embodiments provided herein involving taking an action if a total tardiness risk value exceeds a threshold tardiness risk value, the action may be selected from the group consisting of: contacting the patient, assigning the patient a new scheduled appointment time, and assigning a different patient an appointment at the patient's original scheduled appointment time.

Optionally, in embodiments provided herein involving taking an action based on the value of the total tardiness risk value, the action is selected from the group consisting of: contacting the patient, assigning the patient a new scheduled appointment time, and assigning a different patient an appointment at the patient's original scheduled appointment time.

In embodiments provided herein involving a first quantified tardiness risk value, the first quantified tardiness risk value is derived from historical patient data relating to a first tardiness risk factor.

In embodiments provided herein involving a second quantified tardiness risk value, the second quantified tardiness risk value is derived from historical patient data relating to a second tardiness risk factor.

In embodiments provided herein involving generating a total tardiness risk value from 2, 3, 4, 5, or more quantified tardiness risk values, the values are summed to generate the total tardiness risk value.

In embodiments provided herein involving a tardiness risk factor, the tardiness risk factor may be a real-time or a baseline tardiness risk factor.

In embodiments provided herein involving a total tardiness risk value, the total tardiness risk value is a unit-less value or it is expressed as a time value, such as in hours, minutes, or seconds. In embodiments, the tardiness risk factor comprises accounting for the distance between the patient and the health service center or accounting for the amount of time until the patient's scheduled appointment.

In embodiments provided herein involving a tardiness risk factor, the tardiness risk factor is obtained from a computing device on, in, or adjacent to a patient. The computing device may be, for example, a smartphone or personal computer.

In embodiments provided herein involving a patient having an appointment at a health service center, a sample may be obtained from the patient at the health service center.

In embodiments provided herein involving a patient being assigned a new scheduled appointment time or a different patient is assigned to an appointment at a patient's original scheduled appointment time, and the method may further comprise displaying the patient's new scheduled appointment time or the name of the name of the different patient assigned to the appointment at the patient's original scheduled appointment time on a display at the health service center.

Optionally, methods provided herein may comprise receiving by a health service center computer system comprising a processor information indicating that a patient has brought an NFC-enabled device into close proximity with an NFC tag in a display in a health service center. Such information may include, for example, the time of day that the patient brought the NFC-enabled device into close proximity with the NFC tag in the display at the health service center.

In embodiments, provided herein is a method of managing a patient's arrival at a health service center, the method comprising receiving by a health service center computer system information indicating that a patient has brought an NFC-enabled device into close proximity with an NFC tag in a display in the health service center. Optionally, such methods may further comprising comparing by a processor in the health service center computer system: i) a scheduled appointment time for the patient to ii) the time of day that the patient brought the NFC-enabled device into close proximity with the NFC tag in the display at the health service center. Optionally, the method may further comprise taking an action. Optionally, the action may comprise entering the patient into a service queue at the health service center or suggesting a new appointment time to the patient. A new appointment may be suggested to the patient, for example, if the patient is more than 5, 10, 15, 20, 30, 45, 60, or 120 minutes tardy for his or her original scheduled appointment.

Optionally, a health service center computer system may comprise a processor which is programmed to perform a method disclosed herein.

In embodiments, provided herein is a method of managing a patient's arrival at a health service center, the method comprising receiving by a health service center computer system information indicating that a computing device maintained by the patient is within a selected distance of the health service center. Optionally, the patient has a scheduled appointment at a time at the health service center. Optionally, the selected distance is no greater than 1 mile, ½ mile, ¼ mile, 500 feet, 400 feet, 300 feet, 200 feet, 100 feet, 50 feet, or 10 feet.

In embodiments, provided herein is a system for managing appointments, comprising: a health service center computer system, wherein the health service center computer system comprises a processor which is programmed to perform a method provided herein, and a user device, wherein the is operatively connected to the health service center computer system. Optionally, the user device is a health service center terminal, wherein the health service center terminal is located in a health service center. Optionally, the health service center terminal comprises a display. Optionally, the health service center terminal comprises a keyboard. Optionally, the health service center computer system and the user device are operatively connected via a local network. Optionally, the health service center computer system and the user device are operatively connected via the Internet.

In embodiments, a system provided herein comprises a display containing a NFC tag, wherein the display is located in a health service center.

In embodiments, provided herein is a non-transitory computer-readable medium comprising machine-executable code for implementing a method provided herein. In embodiments, the medium is in a server or a hard drive.

In embodiments, methods, devices, and systems for scheduling appointments are provided. In embodiments, methods, devices, and systems for scheduling appointments for medical services are provided. In embodiments, methods, devices, and systems for scheduling appointments for medical testing are provided. In embodiments, methods, devices, and systems for scheduling appointments for commercial services are provided. In embodiments, methods, devices, and systems for scheduling appointments for transfer of materials are provided.

Methods, devices, and systems for scheduling a plurality of appointments are provided. In embodiments, methods, devices, and systems for scheduling a plurality of appointments for medical services are provided. In embodiments, methods, devices, and systems for scheduling a plurality of appointments for medical testing are provided. In embodiments, methods, devices, and systems for scheduling a plurality of appointments for commercial services are provided. In embodiments, methods, devices, and systems for scheduling a plurality of appointments for transfer of materials are provided.

Methods, devices, and systems for rescheduling appointments are provided. In embodiments, methods, devices, and systems for rescheduling appointments for medical services are provided. In embodiments, methods, devices, and systems for rescheduling appointments for medical testing are provided. In embodiments, methods, devices, and systems for rescheduling appointments for commercial services are provided. In embodiments, methods, devices, and systems for rescheduling appointments for transfer of materials are provided.

Methods, devices, and systems for rescheduling a plurality of appointments are provided. In embodiments, methods, devices, and systems for rescheduling a plurality of appointments for medical services are provided. In embodiments, methods, devices, and systems for rescheduling a plurality of appointments for medical testing are provided. In embodiments, methods, devices, and systems for rescheduling a plurality of appointments for commercial services are provided. In embodiments, methods, devices, and systems for rescheduling a plurality of appointments for transfer of materials are provided.

In embodiments, methods are disclosed herein for scheduling an appointment for an appointee, wherein the method comprises: Selecting a date and time for an appointment for the appointee to arrive at a designated location, and at a time within a designated time period; and then determining if one or more appointments scheduled for a time prior to said appointment time are running late; or determining if weather or a traffic situation relevant to the appointee's arrival on time for the appointment exists which might likely result in the appointee's late arrival; or determining if a health pattern exists which might likely result in a) a greater than usual number of prior appointments, including walk-in appointments, or b) a greater than usual risk that prior appointments will take longer times than usual, and so that a greater than usual risk exists that prior appointments may impact the appointee's appointment; or determining if the appointee's prior history suggests that the appointee may be late for the appointment; or determining if the work history of personnel involved with the appointment, or involved with appointments scheduled for time prior to the appointment, suggests that said personnel involved may not be ready for the appointment at the appropriate time; or determining if supplies or services required for the appointment may not be available or may not be ready in time for the appointment; or determining if a factor exists which might likely result in a greater than usual risk that prior appointments may impact the appointee's appointment; wherein said determining is performed at a time prior to said selected time on the date of the appointment; and optionally, re-scheduling said appointee's appointment to a later time.

Applicants provide further methods, further comprising a step of notifying said appointee of said rescheduled appointment time prior to the scheduled appointment time.

Applicants provide further methods, further comprising a step of receiving a message from said appointee, wherein if said message indicates the appointee may be late for the appointment, then rescheduling the appointment time to a later time.

Applicants provide further methods, further comprising a step of rescheduling personnel work schedules upon rescheduling the appointment time to a later time.

In embodiments, systems and methods are provided for adjusting an appointee's appointment time to an earlier time if the appointee's level of illness is increasing, or to a later time if the appointee's level of illness is decreasing, and optionally, if there is demand by others for the appointee's appointment time.

In embodiments, systems and methods are provided for adding additional appointment times for an appointee in view of test results or symptoms exhibited by the appointee.

In embodiments, non-transitory tangible computer readable media comprising machine-executable code for implementing methods provided herein may be provided as a stand-alone and transportable product (e.g. a DVD, flash drive, magnetic tape, or other form of removable/insertable computer-readable media), such that a program or software stored thereon can be loaded onto one or more different computers, servers, or other computing devices, in order to implement one or more methods provided herein (or elements thereof). In other embodiments, non-transitory tangible computer readable media comprising machine-executable code for implementing methods provided herein may be provided as part of a computing system involving multiple components (e.g. a server or personal computer). In embodiments, a user may interact with software on a server via a client application running on a user device, which is coupled to the server via a network. For example, the software may include a WWW-based interface to allow a remote user/client to access and view appointment-related information. In embodiments, software running on a server may provide certain features to a user (e.g. a WWW-based interface), while performing various processes/operations on the server (e.g. calculation of a total tardiness risk value).

In embodiments, provided herein is an appointment-scheduling or check-in apparatus taking the form of a machine readable storage medium (e.g., hard disk, CD, or other medium) (or multiple media) which contains a set of software instructions for execution by a processor for performing methods provided herein.

In embodiments, methods provided herein may be implemented using hardware, software, or a combination thereof. In embodiments, software code may be implemented using one or more processors, which may be distributed between one or more computing devices.

In embodiments, processors described herein may be programmed to perform methods described herein. Accordingly, in embodiments, a processor provided herein may have a specialized function to perform a method provided herein. Such processors may be part of, for example, a server, computing device, or health service center computer system provided herein.

Other goals and advantages of the invention will be further appreciated and understood when considered in conjunction with the following description and accompanying drawings. While the following description may contain specific details describing particular embodiments of the invention, this should not be construed as limitations to the scope of the invention but rather as an exemplification of preferable embodiments. For each aspect of the invention, many variations are possible as suggested herein that are known to those of ordinary skill in the art. A variety of changes and modifications can be made within the scope of the invention without departing from the spirit thereof.

### BRIEF DESCRIPTION OF THE DRAWINGS

In the drawings,

FIG. 1A shows a schematic of exemplary components of a system provided herein; FIG. 1B shows a schematic of exemplary components of a health service center computer system.

FIG. 2 shows a schematic of exemplary steps of a method provided herein.

FIG. 3 shows a schematic of exemplary steps of a method provided herein.

FIG. 4 shows an exemplary screenshot on a smartphone of part of a method provided herein.

FIGS. 5A and 5B show exemplary screenshots on a smartphone of part of a method provided herein.

FIG. 6 shows an exemplary screenshot on a smartphone of part of a method provided herein.

FIGS. 7A through 7I show exemplary screenshots on a smartphone of part of a method provided herein.

FIGS. 8A through 8F show exemplary screenshots on a smartphone of part of a method provided herein.

FIGS. 9A through 9K show exemplary screenshots on a smartphone of part of a method provided herein.

FIGS. 10A through 10D show exemplary screenshots on a smartphone of part of a method provided herein.

It is noted that the drawings and elements therein are not necessarily drawn to shape or scale. For example, the shape or scale of elements of the drawings may be simplified or modified for ease or clarity of presentation. It should further be understood that the drawings and elements therein are for exemplary illustrative purposes only, and not be construed as limiting in any way.

### DETAILED DESCRIPTION

Provided herein are systems and methods for appointment scheduling and check-in. Various features described herein may be applied to any of the particular embodiments set forth below or for any other types of system or method for appointment scheduling or check-in. Systems and methods described herein may be applied as a standalone system or method, or as part of an integrated system or method. It shall be understood that different aspects of the disclosed systems and methods can be performed individually, collectively, or in various combinations with each other.

Systems and methods provided herein may relate to making appointments, modifying appointment times, or checking in for an appointment. In embodiments, systems and methods provided herein are described relative to a patient's original scheduled appointment time. A patient may obtain an original scheduled appointment time through any appropriate mechanism, such as by being assigned an appointment time by a health service center employee, or by requesting an appointment time (e.g. by phone or on-line). A patient having a scheduled appointment time typically has the appointment scheduled at a particular health service center (i.e. at a particular address).

As used herein, a “health service center” refers to any location that may provide a health-related service for a subject (e.g. treatment, testing, monitoring, counseling, etc.). Health service centers may include, for example, hospitals, health care providers' offices, pharmacies, clinics, or retail locations. In embodiments, a “health service center” may be an establishment which provides various functions besides health-related services, such as a retail location (e.g. a grocery store, drug store, or shopping mall) which contains a kiosk or other area for the performance of health-related services. In embodiments, health service centers may accept patients only on a walk-in basis, only on an appointment basis, or on both a walk-in and an appointment basis. In embodiments, a health service center is a patient service center. In embodiments, a sample may be collected from a patient at a health service center. The sample may be, for example, blood, saliva, urine, mucus, or a portion thereof. In embodiments, a sample collected from a patient may have a volume of less than 1000, 500, 400, 300, 200, 100, 90, 80, 70, 60, 50, 40, 30, 20, or 10 microliters. In embodiments, a sample collected from a patient may be processed at a health service center or elsewhere by a sample processing device having any of the features described in, for example, International Application No. PCT/US2014/016997, U.S. Pat. No. 8,088,593, or U.S. Pat. No. 8,435,738, each of which is hereby incorporated by reference in its entirety for all purposes.

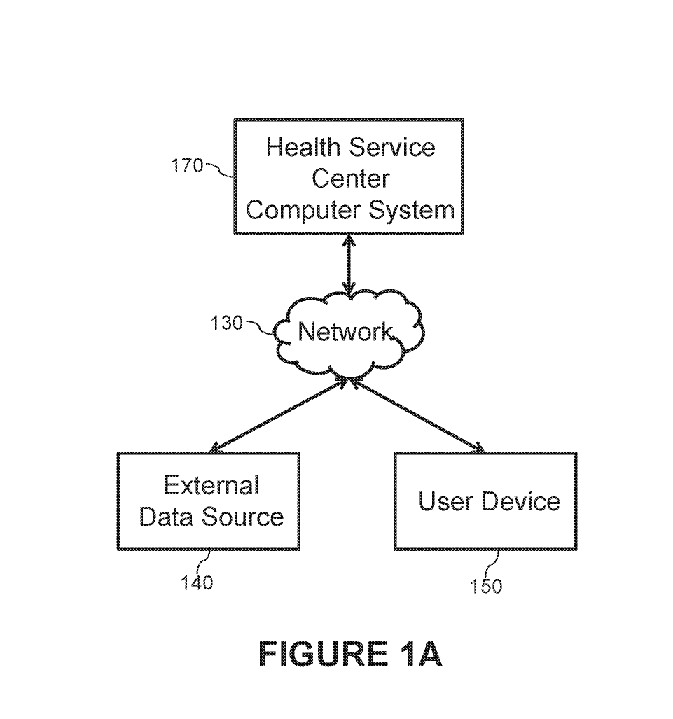

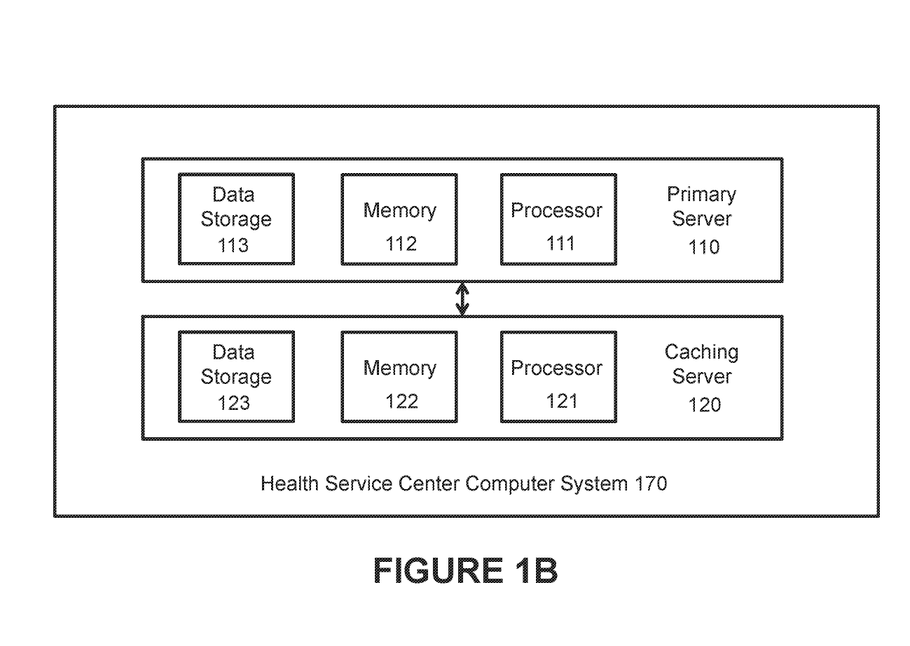

Embodiments of systems and methods provided herein may be described with reference to FIG. 1, which provides an exemplary schematic of possible components of systems provided herein. A health service center computer system 170 may perform various appointment-related functions, such as storing or processing appointment-related information. A health service computer system may have any configuration and may contain any combination of hardware and software as appropriate for performing methods described herein. A health service center computer system may contain, for example, one or more servers, such as a primary server and a caching server. A health service center computer system 170 may be operably connected to an external data source 140 or a user device 150. An external data source 140 may be any computing device which may store, transmit, receive, or gather data that may be accessed by or sent to a health service center computer system (e.g. a hard drive, server, personal computer, or smartphone). The user device 150 may be any computing device with which a user may access a health service center computer system and with which a user may, for example, review, input, or change appointment-related information (e.g. a smartphone or personal computer). Although FIG. 1 shows only a single external data source 140 and user device 150 operatively connected to a health service center computer system 170, any number of external data sources 140 and user devices 150 may be operatively connected to a health service center computer system 170 (e.g. at least 2, 3, 4, 5, 10, 50, 100, 1000, 10,000, or more). In embodiments, a health service center computer system 170 may be connected to an external data source 140 or a user device 150 via a network 130. The network 130 may be any structure which can support the operable connection of and data transfer between two or more computing devices, such as a local area network (LAN) or a wide area network (WAN), and may include, for example, an intranet or the Internet. Also, although FIG. 1 depicts the external data source 140 and user device 150 being operatively connected to the health service center computer system 170 via a network 130, in embodiments, an external data source 140 or user device 150 may connect directly to a health service center computer system 170 (i.e. not through a separate network).

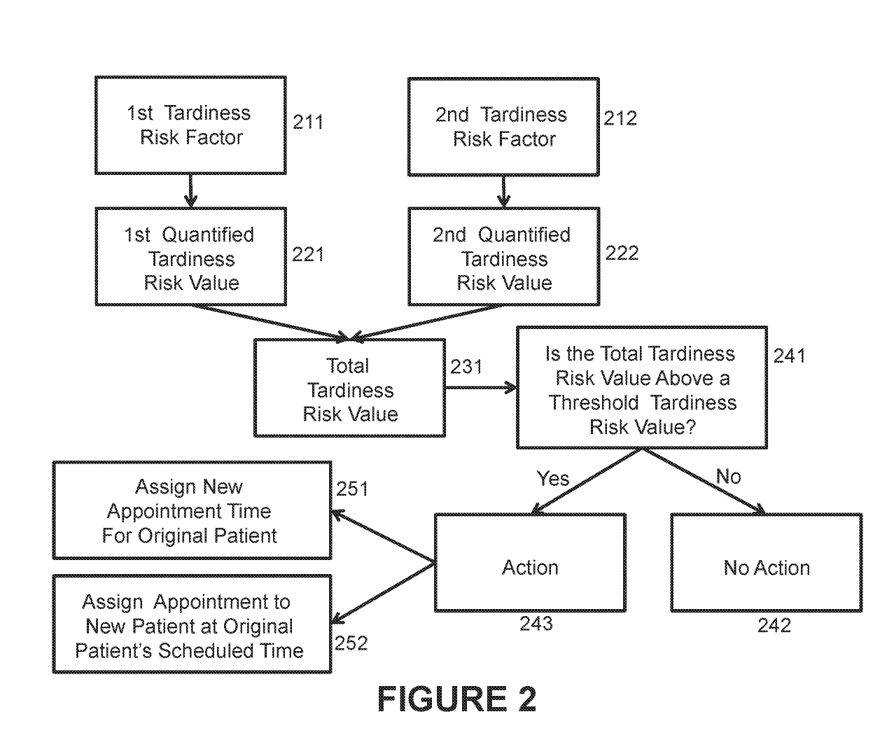

Exemplary steps of embodiments of methods provided here may be described with reference to FIG. 2. Some or all of the steps depicted in FIG. 2 may be performed by hardware or software contained within or operatively connected to a health service center computer system described herein.

For a patient having a scheduled appointment time, one or more tardiness risk factors may be obtained. In embodiments, at least a first tardiness risk factor 211 and a second tardiness risk factor 212 may be obtained. Although FIG. 2 depicts obtaining two tardiness risk factors, any number (e.g. 1, 2, 3, 4, 5, 10, or more) of tardiness risk factors may be obtained with systems and methods provided herein.

As used herein, a “tardiness risk factor” refers to any factor which may influence the likelihood of a patient arriving at a health service center on time (or tardy) for an appointment, such as the amount of traffic on one or more roads between the patient and the health service center, or the patient's history of tardiness for other appointments. In embodiments, tardiness risk factors may be classified as either “real-time” or “baseline” tardiness risk factors.

Real time tardiness risk factors are factors which are evaluated in a time period immediately preceding a patient's scheduled appointment time. Generally, real-time tardiness risk factors are evaluated at one or more time points 4 hours or less before the patient's scheduled appointment time, such as less than 4 hours, 3 hours, 2 hours, 1 hour, 45 minutes, 30 minutes, 20 minutes, 15 minutes, 10 minutes 5 minutes, 4 minutes, 3 minutes, 2 minutes, or 1 minute before the patient's scheduled appointment time. In some situations, however, real-time tardiness risk factors may be evaluated at one or more time points between 24 hours and 4 hours before the patient's scheduled appointment time, such as less than 6 hours, 8 hours, 12 hours, 16 hours, or 24 hours before the patient's scheduled appointment time. Real time tardiness risk factors may include, for example, the location of the patient, the geographic distance between the patient and the health service center where the patient has an appointment, the estimated driving time for the predicted fastest driving route between the location of the patient and the health service center, the estimated driving time for the predicted fastest driving route between the location of the patient's home address and the health service center, traffic conditions on one or more roads on the predicted fastest driving route between the location of the patient and the health service center, traffic conditions on one or more roads on the predicted fastest driving route between the location of the patient's home address and the health service center, conditions on one or more public transportation system routes between the patient and the health service center, conditions on one or more public transportation system routes between the patient's home address and the health service center, weather conditions at the location of the health service center, weather conditions at the location of the patient, weather conditions at the location of the patient's home address, or tardiness amounts of other patients at a patient of interest's health service center in a time frame immediately preceding the patient of interest's scheduled appointment time. Many real time tardiness risk factors are evaluated in the context of the amount of time until a patient's scheduled appointment time (or, if the patient is already tardy, the amount of time after the patient's scheduled appointment time). For example, an evaluation of the location of a patient may also include an evaluation of the time until or after the patient's scheduled appointment. In another example, an evaluation of tardiness amounts of other patients at a patient of interest's health service center in a time frame immediately preceding the patient of interest's scheduled appointment time may include an evaluation of the time until the patient of interest's scheduled appointment.

Baseline tardiness risk factors are factors which are available well before a patient's scheduled appointment, and which typically involve historical data relevant to a patient's appointment timing. Baseline tardiness risk factors may include, for example, the patient's history of tardiness for previous appointments at health service centers, other patients' history of tardiness for appointments at health service centers at the time of the patient's appointment (e.g. other patients' history of tardiness at a given time of the day, day of the week, or both), and in the event the patient has an appointment to undergo a specific laboratory procedure, one or more other patients' history of tardiness for appointments for the patient of interest's same specific laboratory procedure. Any of these factors may be evaluated for the specific health service center where the patient's appointment is located, or for one or more other health service centers. For example, information may be obtained for one or more other health service centers which may share one or more similar characteristics of the specific health service center where the patient's appointment is located, such as: located in the same part of a town, located in the same city, located in the same state, located in the same country, having of similar size, having similar physical features (e.g. no parking lot), etc. In embodiments, information may be obtained for appointments at one or more other health service centers which are not selected based on similar characteristics of the specific health service center where the patient's appointment is located (e.g. information may be obtained from any grouping of or type of health service center). When information is obtained for two or more data points (e.g. from two or more patients or two or more events from the same patient), the data may be averaged or otherwise integrated to generate a composite value incorporating multiple data points. Baseline tardiness risk factors may be available, for example, at least 8 hours, 12 hours, 24 hours, 2 days, 1 week, 2 weeks, 3 weeks, 1 month, or 2 months before a patient's scheduled appointment. Baseline tardiness risk factors may be determined, for example, based on events that occurred at least 8 hours, 12 hours, 24 hours, 2 days, 1 week, 2 weeks, 3 weeks, 1 month, or 2 months before a patient's scheduled appointment.

When one or more tardiness risk factors are obtained with a method provided herein, a tardiness risk factor may be a real time tardiness risk factor or a baseline tardiness risk factor. In embodiments, when two are more tardiness risk factors are obtained, all of the risk factors may be real time tardiness risk factors, all of the risk factors may be baseline tardiness risk factors, or the risk factors may be at least one real time and at least one baseline tardiness risk factor. Tardiness risk factors may be obtained from one or more sources, such as an external data source or a data storage unit. For example, an external data source may be a GPS-enabled smart phone which provides a real time tardiness risk factor of the current location of the patient. In another example, a data storage unit may be a data storage unit in a server of the health service center computer system, which provides a baseline tardiness risk factor of historical data of other patients' average tardiness for appointments at the same time of day and day of the week as the patient's scheduled appointment. In embodiments, for a patient having a scheduled appointment, tardiness risk factors may be obtained from 1, 2, 3, 4, 5, 6, 7, 8, 9, 10, 15 or more sources (e.g. if the risk factors are from three different sources, they may be from a patient's smart phone, an external data source containing traffic information, and a data storage unit containing the patient's history of tardiness for other appointments). In embodiments in which a total of at least three tardiness risk factors are obtained from two or more sources, from at least one of the sources two or more tardiness risk factors may be provided. Tardiness risk factors for a patient's scheduled appointment may be provided to a health service center computer system.

Referring again to FIG. 2, the tardiness risk factors may be inputted into a computer program which, with the aid of a processor, converts each tardiness risk factor into a quantified tardiness risk value. For example, a first tardiness risk factor 211 may be converted into a first quantified tardiness risk value 221 and a second tardiness risk factor 212 may be converted into a second quantified tardiness risk value 222. In embodiments, a quantified tardiness risk value may be based on historical patient data relating to a tardiness risk factor. In other embodiments, a quantified tardiness risk value may be based on a calculation or other measurement relating to a tardiness risk factor, and not involve historical patient data. Once quantified tardiness risk values are obtained for each assessed tardiness risk factor, the quantified tardiness risk values may be combined to generate a total tardiness risk value 231. In some situations, one or more decisions may be made based on the total tardiness risk value 231, such as whether or not to take an action, or what action to take. For example, in embodiments, if a total tardiness risk value 231 is a certain value, the patient's appointment time may be adjusted by a corresponding amount. The relationship between a total tardiness risk value 231 and the amount of time by which to adjust the patient's appointment time (e.g. 10 minutes later, 15 minutes later, etc.) may be based on a formula which correlates total tardiness risk value to preferred appointment time adjustment amounts. This formula may be based, for example, on historical data relating to a tardiness risk value 231 and the average amount of tardiness of previous patients with a given total tardiness risk value. In certain embodiments, the total tardiness risk value may be compared with a threshold tardiness risk value 241. In embodiments, if the total tardiness risk value does not exceed a threshold tardiness risk value, no action may be taken 242. In contrast, if the total tardiness risk value exceeds the threshold tardiness risk value, an action may be taken 243. Actions that may be taken may include, for example, assigning a new appointment time for the patient (the “original patient”) 251 or assigning a new patient to an appointment at the original patient's scheduled time 252.

Real Time Tardiness Risk Factors

For systems and methods provided herein, real time tardiness risk factors may be obtained from a variety of sources, such as one or more external data sources 140 or user devices 150.

The amount of time until a patient's scheduled appointment may be determined, for example, by a server of a patient service center computer system or provided to a server from a separate computing device. Typically, computing devices have a clock or clock function in order to keep track of time. A computing device may determine the amount of time until a patient's scheduled appointment time by determining the difference in time between the present time and the patient's scheduled appointment time. This may be determined, for example, by a processor executing software instructions for determining the amount of time until a patient's scheduled appointment time.

Location data of a patient may include, for example, longitude and latitude data, or longitude, latitude, and altitude data. In some cases, location data is collected using wireless triangulation. In an example, wireless triangulation may use IEEE 802.11 standards to determine the location of a subject. In other situations, location data may be collected using a global positioning system (GPS). The global positioning system may use signals from 2, 3, or 4 or more satellites to determine the location of a computing device having a global positioning system.

A wide variety of different computing devices may be used to collect location data of a patient. For example, an external data source 140 on or associated with a patient containing a GPS receiver (e.g. a GPS-enabled phone, watch, laptop computer, or tablet computer) may collect location data of a patient. Location data of a patient may be provided to health service center computer system 170. In embodiments, location data of a patient obtained by an external data source 140 may be directed to health service center computer system 170 via a software application running on the external data source 140 (e.g. an “app”). In embodiments, patient location data obtained by an external data source 140 may be directed to a health service center computer system by a patient uploading his or her location data to the health service center computer system at one or more intervals. In embodiments, a patient may upload his or her location data to the health service center computer system upon receiving a reminder from a health service center computer system requesting the patient to upload his or her location data to the server.

Software applications may be loaded onto an external data source 140, for example, during manufacturing of the external data source 140 or at a later point. For example, a user may load a software application onto the external data source 140, for example, by downloading it onto the external data source 140 from the health service center computer system. A software application may be configured to obtain location data from an external data source 140 configured to obtain location data (e.g. a GPS-enabled smart phone), and to communicate the location data to the health service center computer system. In some embodiments, the application may be designed to provide features and services for a user which are associated with the health service center computer system 170. For example, the application may permit a user to input personal information for storage with a health service provider, to request an appointment for a service, or to review laboratory test results. In embodiments, a user who has installed the application on an external data source 140 may select whether or not to have the application collect location data of the external data source 140.

In embodiments, location data may also or alternatively be obtained with other devices which may obtain information relating to the location of an individual, such as street cameras, cameras on computers, tracking devices on automobiles, cell phone towers, or wide area information server (WAIS). Some devices may record driving habits. Location can also be inferred from multiple activities that suggest location, such as logging into computers/networks having a defined location, or by personal behavior data such as credit card purchases at a particular store location.

Real time tardiness risk factors such as the geographic distance between a patient and the health service center where the patient has an appointment or the estimated driving time for the predicted fastest driving route between the location of the patient and the health service center may be determined with the aid of any one or more of various programs available in the art. For example, relevant WWW-based programs include MapQuest, Yahoo Maps, Google Maps, and Rand McNally Maps. In other embodiments, non-WWW based programs (e.g. a software program on a data storage unit operatively connected to or directly linked to a server of a health service center computer system) may be used for determining geographic-distance related calculations relating to the distance between the patient and the health service center. In embodiments, a health service center computer system provided herein performs an operation wherein the health service center computer system receives geographic information regarding a specific patient having a scheduled appointment (i.e. geolocation data of the patient's current location), and then communicates with a WWW-based or other program to determine, for example, the distance between the patient and the health service center or the estimated driving time between the patient and the health service center. In embodiments, programs for determining the estimated driving time between two locations may provide multiple time estimates based on multiple different possible routes. A health service center computer system may be configured, for example, to select the most rapid route out of multiple possible routes, or to perform other operations, such as to average the time of the fastest 2, 3, 4 or more routes.

Real time tardiness risk factors such as traffic conditions on one or more roads on a predicted fastest driving route between the location of a patient and a health service center or traffic conditions on one or more roads on a predicted fastest driving route between the location of a patient's home address and a health service center may be determined, for example, with the aid of various programs known in the art for the determination of traffic conditions. For example, relevant WWW-based applications include here.com, traffic.com, localconditions.com, and beatthetraffic.com. Other WWW-based providers of traffic conditions may also be used with systems and methods provided herein. In other embodiments, non-WWW based applications (e.g. direct images or videos of roadways, radio-based reports, TV-based reports) may be used for determining traffic information relevant to the location of a patient or a health service center. In embodiments, a health service center computer system performs an operation wherein the system receives traffic information relating to a driving route of interest, and uses the information as part of a determination of a tardiness risk value.

Real time tardiness risk factors such as conditions on one or more public transportation systems between a patient and a health service center or between a patient's home and a health service center may be determined, for example, with the aid of various programs known in the art for the determination of conditions on public transportation systems. For example, relevant WWW-based applications include mta.info, wmata.com, bart.com, transitchicago.com and tfl.gov.uk. Other WWW-based providers of public transportation system conditions may also be used with systems and methods provided herein. For example, many transportation systems in cities and regions throughout the world provide updates on a WWW-based site as to current conditions on their transportation system. In other embodiments, non-WWW based applications (e.g. direct images or videos of public transportation systems, radio-based reports, TV-based reports) may be used for determining public transportation system information relevant to the location of a patient or a health service center. In embodiments, a health service center computer system performs an operation wherein the health service center computer system receives public transportation system information relating to a transit path of interest, and uses the information as part of a determination of a tardiness risk value.

Real time tardiness risk factors such as weather conditions at the location of the health service center, the location of the patient, or the location of the patient's home address may be determined, for example, with the aid of various programs known if the art for the determination of weather conditions. For example, relevant WWW-based applications include weather.com, wunderground.com, weather.gov, and accuweather.com. Other WWW-based providers of weather conditions may also be used with systems and methods provided herein. In other embodiments, non-WWW based applications (e.g. direct weather data measurements, radio-based reports, TV-based reports) may be used for determining weather information relating to a location. In embodiments, a health service center computer system performs an operation wherein the health service center computer system receives weather information regarding a location of interest (e.g. the location of a patient or of a health service center), and uses the information as part of a determination of a tardiness risk value.

Real time tardiness risk factors such as the tardiness amounts of other patients at a patient of interest's health service center in a time frame immediately preceding the patient of interest's scheduled appointment time may be determined, for example, by obtaining data from the patient of interest's health service center. In embodiments, one or more external data sources located a patient of interest's health service center (e.g. a personal computer at the health service center) may be operably connected with a health service center computer system, and may provide information to the health service center computer system regarding the timeliness of arrival for appointments of other patients at the patient of interest's health service center in a time frame immediately preceding the patient of interest's scheduled appointment time. Data relating to tardiness amounts of other patients at a patient of interest's health service center in a time frame immediately preceding a patient of interest's scheduled appointment may be relevant to the tardiness risk for the patient of interest due to the fact that, for example, if conditions near the health service center are such that multiple other patients have been delayed for an appointment (e.g. due to bad weather, slow traffic, etc. around the health service center), the patient of interest may also have an increased likelihood of tardiness.

Baseline Tardiness Risk Factors

Baseline tardiness risk factors may be obtained from a variety of sources, such as one or more external data sources or a data storage unit in or operatively connected to a server of a health service center computer system. In embodiments, a baseline tardiness risk factor may include information that is originally obtained from one or more external data sources, and then compiled or stored in a different location than from where it originated (e.g. it may be stored in a different external data source or a data storage unit). For example, in the case of the patient's history of tardiness for previous appointments at a health service center, this information may originally be obtained from an external data source or user device located at a health service center. The external data source or user device may be, for example, a computer operated by the health service center which is used for appointment-related transactions. The external data source or user device may have recorded when the subject checked-in for one or more previous appointments [e.g. personnel at the health service center may have manually entered into the external data source or user device the patient's time of arrival at the health service center, or the patient may have checked-in automatically using a WWW-based application or a NFC-based process (described further elsewhere herein)]. In embodiments, information obtained by an external data source at a health service center may be sent from the external data source to a health service center computer system, or sent to a different external data source or a data storage unit, where the information may be stored and may be available to a health service center computer system. Such information may be used as a (or part of a) baseline tardiness risk factor. Information regarding baseline tardiness risk factors may commonly be obtained at health service centers, which may obtain data regarding particular patients or patient tardiness trends for particular days, times, or tests. In embodiments, external data sources which are not located in health service centers may be a source of data for the determination of baseline tardiness risk factors. For example, in embodiments, patients may check-in to an appointment at a health service center via a WWW-based application, which is operably connected to a health service center computer system. Once a patient checks in to an appointment, this information may be directly sent to a health service center computer system or other device which can store or process appointment-related information. This check-in information may thus be sent directly sent to a system or device for storing or processing appointment-relating information (e.g. a health service center computer system), without passing through an external data source located at a patient service center.

Converting a Tardiness Risk Factor into a Quantified Tardiness Risk Value

A tardiness risk factor may be inputted into a computer program which, with the aid of a processor, converts the tardiness risk factor into a quantified tardiness risk value. For example, a health service center computer system may obtain one or more tardiness risk factors for a patient's scheduled appointment, as described above. According to a computer program, a processor in a server the health service center computer system may obtain quantified tardiness risk values for each of the tardiness risk factors from a computer memory device. As used herein, a “computer memory device” refers to any computer structure for storing information, and includes, for example, a data storage unit and memory of a server, as well as structures outside of a server (e.g. a stand-alone hard drive, removable memory device, etc.). A computer memory device may contain one or more databases which store quantified tardiness risk values corresponding to tardiness risk factors. In embodiments, a quantified tardiness risk values may be derived from historical patient data relating to each tardiness risk factor. In other embodiments, a quantified tardiness risk value may be based on a calculation or other measurement relating to a tardiness risk factor, and not involve historical patient data. For example, in embodiments, a quantified tardiness risk value relating to a tardiness risk factor may be based solely on how far away a patient is from the site of his or her health service center at a given time prior to his or her appointment, and not involve historical patient data. In embodiments, a quantified tardiness risk value for a tardiness risk factor may take into account both a given a tardiness risk factor and the amount of time until (or after) a patient's scheduled appointment time. For example, for a tardiness risk factor of traffic conditions on one or more roads on the predicted fastest driving route between the location of the patient and the location of the health service center where the patient has an appointment, different quantified tardiness risk values may be provided for a given traffic condition (e.g. the risk factor of“heavy traffic” may have different quantified tardiness risk values depending on whether the condition is evaluated, for example, 1 hour before, 30 minutes before, or 5 minutes before a patient's scheduled appointment time). If a quantified tardiness risk value is generated based on historical data, a quantified tardiness risk value may be generated based on historical data from, for example, at least 1, 2, 3, 4, 5, 10, 15, 20, 50, 100, 200, 300, 500, 1000, 5000, or 10,000 patients. Optionally, quantified tardiness risk values can be refined and adjusted over time, as more data is collected. For instance, initially, quantified tardiness risk values for patient tardiness relating to different traffic conditions may be generated based on data from at least 50 patients for each of a number of different traffic conditions (e.g. heavy rain, heavy snow, light rain, fog, sunshine, etc.). These quantified tardiness risk values may be used by a system or method provided herein for a period of time while further patient data for additional patients is collected. Continuing with this same example, after data from at least 100 patients for one or more of the different traffic conditions is obtained, the quantified tardiness risk values for the different traffic conditions may be adjusted, based on the additional gathered data. In embodiments, with systems and methods provided herein, quantified tardiness risk values may be continuously updated or updated at regular intervals (e.g. every day, week, month, or year, or after every 1, 2, 3, 5, 10, 15, 20, 50, 100 patients) as additional data is obtained. Quantified tardiness risk values may be generated on any numerical scale as desired and appropriate for a selected algorithm. For instance, quantified tardiness risk values may be whole numbers, fractions, decimal numbers, etc. Also, in embodiments, with systems and method provided herein, a tardiness risk factor may or may not have a non-zero quantified tardiness risk value. For example, in embodiments, every tardiness risk factor has a non-zero quantified tardiness risk value. In other embodiments, some tardiness risk factors have a quantified tardiness risk value of 0. (i.e. if the “tardiness risk factor” is not expected to cause a delay in a patient arriving for a scheduled appointment—for example, a tardiness risk factor of sunny weather with no precipitation may have a quantified tardiness risk value of 0, since this weather condition is not expected to cause a delay for a patient arriving for a scheduled appointment) Also, a quantified tardiness risk value may be based on a selected definition of “tardiness”. For example, a patient may be considered tardy if he or she is does not arrive by, for example, at least 10, 5, 3, 2, or 1 minutes before, or 1, 2, 3, 4, 5, 10, 15, or 20 minutes after, or by the scheduled appointment time. By collecting multiple data points relating patients' appointments (e.g. scheduled appointment time, exact time of patient arrival for scheduled appointment, weather conditions, day of the week, etc.), historical data can be analyzed and used to generate quantified tardiness risk values based on conditions of interest. Exemplary representations of exemplary databases containing quantified tardiness risk values corresponding to tardiness risk factors are provided below in Tables 1 and 2.

Table 1 shows exemplary quantified tardiness risk values corresponding to weather-related tardiness risk factors. The weather conditions may refer to conditions at any of a number of different locations, such as for example, the health service center, the location of a patient, or the location of patient's home. Also, the quantified tardiness risk values may be for a given time range relative to a patient's scheduled appointment time. For instance, the quantified tardiness risk values in the chart below may be, for example, for weather conditions at 30 to 15 minutes before the patient's scheduled appointment time. If the weather conditions are obtained, for example, at 45 to 30 or 15 to 0 minutes before the patient's scheduled appointment time, some or all of the quantified tardiness risk values may be different. The quantified tardiness risk values for each risk factor (including both the risk factor itself, as well as the time period relative to a patient's scheduled appointment time) may be determined based on historical patient data. For instance, a health service center computer system provided herein may collect data on the tardiness of patients for appointments during different weather conditions, and based on this data, generate quantified tardiness risk values for various weather conditions. This tardiness risk factor is a real-time tardiness risk factor, and as, such, the corresponding quantified tardiness risk values typically may vary based on the time until the patient's scheduled appointment. While exemplary values are provided in this table, any suitable value as determined by a user of a system or method provided herein may be used. Also, in embodiments, a quantified tardiness risk value is not based on historical patient data, and is instead, based on another metric.

TABLE 1

| Tardiness | | | | | Light | Moderate | Heavy |
|---|---|---|---|---|---|---|---|
| Risk Factor - | | | | | Snow, | Snow, | Snow, |
| Weather | No | Light | Moderate | Heavy | Sleet, or | Sleet, or | Sleet, or |
| Conditions | Precipitation | Rain | Rain | Rain | Hail | Hail | Hail |
| Quantified | 0 | 0.1 | 0.2 | 0.3 | 0.25 | 0.35 | 0.45 |
| Tardiness | | | | | | | |
| Risk Value | | | | | | | |

Table 2 shows exemplary quantified tardiness risk values corresponding to appointment date and time-related tardiness risk factors. The table shows exemplary quantified tardiness risk values for appointments at 4 PM, for each day of the week. The quantified tardiness risk values may be based on historical patient data. For instance, a system provided herein may collect data on the tardiness of patients for appointments at 4 PM for different days of the week, and based on this data, generate quantified tardiness risk values for appointments at 4 PM each day of the week. This tardiness risk factor is a baseline tardiness risk factor, and as, such, the corresponding quantified tardiness risk values typically will not vary based on the time until the patient's scheduled appointment. While exemplary values are provided in this table, any suitable value as determined by a user of a system or method provided herein may be used.

TABLE 2

| Tardiness | | | | | | | |
|---|---|---|---|---|---|---|---|
| Risk Factor - | | | | | | | |
| 4 PM | | | | | | | |
| Appt. | Monday | Tuesday | Wednesday | Thursday | Friday | Saturday | Sunday |
| Quantified | 0.1 | 0 | 0 | 0 | 0.3 | 0.2 | 0.2 |
| Tardiness | | | | | | | |
| Risk Value | | | | | | | |

In embodiments, a quantified tardiness risk value may not have any unit of measurement. For instance, a quantified tardiness risk value may be a unit-less value which may be subsequently converted into a value having a unit of measurement (e.g. minutes or seconds). In other embodiments, a quantified tardiness risk value may have a unit of measurement. For example, the quantified tardiness risk value may be provided in hours, minutes, seconds, or other unit. A quantified tardiness risk value may be a quantified estimated tardiness amount, and be a value having a unit of time.

In embodiments, a quantified tardiness risk value may be a probability value (e.g. a “quantified tardiness risk probability”). A quantified tardiness risk probability may be used in systems and methods provided in the same way as a quantified tardiness risk value described herein (e.g. two or more may be combined to generate a total tardiness risk value). A quantified tardiness risk probability may be provided for a selected time frame relative to a scheduled appointment time. For example, a selected time frame relative to a scheduled appointment time may be no greater than 0, 1, 2, 3, 4, 5, 6, 7, 8, 9, 10, 15, 20, 25, 30, 45, or 60 minutes after a scheduled appointment time. In another example, a selected time frame relative to a scheduled appointment time may be no greater than 0, 1, 2, 3, 4, 5, 6, 7, 8, 9, 10, 15, 20, 25, 30, 45, or 60 minutes before or after a scheduled appointment time. Thus, for example, a quantified tardiness risk probability including a time frame of no greater than 10 minutes after the scheduled appointment time will be the probability value for the tardiness risk factor of the patient not arriving within 10 minutes of the scheduled appointment time.

A quantified tardiness risk probability may be obtained for a tardiness risk factor based on historical data. For example, if a tardiness risk factor is the real-time risk factor of heavy snow, historical data may show, for example, that when the tardiness risk factor is heavy snow, based on this factor, there is a 60% probability that the patient will not arrive within 5 minutes of the scheduled appointment time (i.e. based on this factor, there is a 60% probability of the patient being more than 5 minutes tardy for the scheduled appointment time). In another example, if a tardiness risk factor is the baseline risk factor of the patient being, on average 18 minutes late for previous appointments at the health service center, historical data may show, for example, that based on this factor, there is a 50% probability that the patient will not arrive within 15 minutes of the scheduled appointment time.

Combining Two or More Quantified Tardiness Risk Values into a Total Tardiness Risk Value

In embodiments, if a single tardiness risk factor is obtained, it may be converted into a quantified tardiness risk value. If two or more tardiness risk values are obtained and each converted into a separate quantified tardiness risk value, the two or more quantified tardiness risk values may be combined to generate a total tardiness risk value. The quantified tardiness risk values may be combined into a total tardiness risk value by an algorithm using any one or more suitable operations, such as summation or multiplication. For example, in a situation in which three quantified tardiness risk values are obtained, the three values may be summed to generate a total tardiness risk value [e.g. quantified tardiness risk value #1 (0.2)+quantified tardiness risk value #2 (0.4)+quantified tardiness risk value #3 (0.1)=total tardiness risk value of 0.7]. In embodiments, one or more of the quantified tardiness risk values may be weighted before or during the combination of quantified tardiness risk values into a total tardiness risk value. For example, a quantified tardiness risk value of a first tardiness risk factor may be weighted, for example, 0.5, 1, 1.5, 2, 3, 4, 5, or 10 times the quantified tardiness risk value of a second tardiness risk factor. In embodiments, a quantified tardiness risk value of a real time tardiness risk factor may be weighted, for example, 0.5, 1, 1.5, 2, 3, 4, 5, or 10 times the quantified tardiness risk value of a baseline tardiness risk factor. In embodiments, a quantified tardiness risk value of a baseline tardiness risk factor may be weighted, for example, 0.5, 1, 1.5, 2, 3, 4, 5, or 10 times the quantified tardiness risk value of a real time tardiness risk factor. In embodiments, a total tardiness risk value may be calculated based on a set number of tardiness risk factors (e.g. 2, 3, 4, 5, 6, 7, 8, 9, or 10) or a set of specific tardiness risk factors (e.g. four factors such as: geographic distance between the patient and the health service center, weather conditions at the location of the health service center, the patient's history of tardiness for previous appointments at health service centers, and other patients' history of tardiness for appointments at health service centers at the same time and day of the week of the patient of interest's appointment). The algorithm may be performed by a processor described herein, and stored in a computer-readable medium. In embodiments, if a single tardiness risk factor is obtained and converted into a quantified tardiness risk value, the single quantified tardiness risk value may function as a total tardiness risk value.

Comparing a Total Tardiness Risk Value to a Threshold Tardiness Risk Value

In embodiments, upon the generation of a total tardiness risk value, the total tardiness risk value may optionally be compared with a threshold tardiness risk value. The comparison may be performed, for example, by a processor of a server of a health service center computer system. In embodiments, a threshold tardiness risk value may be selected based on historical patient data relating to total tardiness risk values and the tardiness of patients for scheduled appointments. In embodiments, a threshold tardiness risk value may be modified over time based on historical patient data. For instance, as data is gathered relating to different patients' total tardiness risk value and the likelihood of a given patient having a certain total tardiness risk value being tardy for a scheduled appointment, threshold tardiness risk values may be modified to more accurately reflect historical data. As used herein, “historical data” may be collected at any relevant time period prior to measurement point (e.g. at least 1 day, 1 week, 1 month or 1 year). In embodiments, a threshold tardiness risk value may be selected based on information other than historical patient data. For instance, a threshold tardiness risk value may be based on quantitative information relating to a total tardiness risk value. For example, according to a given algorithm for determining a total tardiness risk value, any patient who is more than 20 miles away from his or her health service center at a time 10 minutes before his or her scheduled appointment at the health service center may have a calculated total tardiness risk value of at least 5 (tardiness risk values may be unit-less). Continuing with the example, a threshold may be set where a total tardiness risk value of, for example, 5 or more triggers an action. In such example, the threshold may be based not on historical patient data but on the information that any patient who is more than 20 miles away from his or her patient service center at a time 10 minutes before his or her scheduled appointment at the patient service center will have a calculated total tardiness risk value of at least 5 (and will certainly not make it to the health service center on time, given that the patient would have to travel at a speed of at least 120 miles per hour to make it to the appointment on time in this circumstance). In this example, the threshold is selected based on a value which represents a situation where a patient essentially cannot make it to the appointment on time, the conclusion being based on the fact that it is generally difficult for humans to perform local travel at a rate of greater than 60 miles per hour.

In embodiments, a threshold tardiness risk value is a value above which there is a significant risk that a patient will be at least a selected period of time tardy. Different threshold tardiness risk values may be selected for different risk levels and different selected period of time for tardiness. For instance, a threshold tardiness risk value may be selected for which if the total tardiness risk value is at least the amount of the threshold tardiness risk value, than there is at least a high risk that a patient will be at least 20 minutes late for his or her appointment. In embodiments, different risk levels and different selected periods of time for tardiness may not be numerically quantified. For instance, a threshold tardiness risk value may be selected above which it is believed there is a “high” risk that a patient will be “seriously” tardy. In such an example, it may not be necessary to assign a quantitative value to the level of risk that the patient will be tardy, or a quantitative value to the degree of tardiness (e.g. number of minutes) that a patient will be tardy. Instead, the threshold tardiness risk value may be selected based upon an empirical or other determination of a value suitable providing an effective point around which (e.g. above or below) to take or not take an action.

In embodiments, a threshold tardiness risk value is a value above which there is a certain probability that a patient will be at least a selected period of time tardy. Different threshold tardiness risk values may be selected for different probabilities and different selected period of time for tardiness. For instance, a threshold tardiness risk value may be selected for which if the total tardiness risk value is at least the amount of the threshold tardiness risk value, than there is at least a 50% probability that a patient will be at least 20 minutes late for his or her appointment. In embodiments, a threshold tardiness risk value may be selected for which if the total tardiness risk value is at least the amount of the threshold tardiness risk value, than there is at least a 10, 20, 30, 40, 50, 60, 70, 80, 90, or 95% probability that a patient will be at least 5, 10, 15, 20, 30, 45, or 60 minutes late for his or her appointment.

When a total tardiness risk value is compared to a threshold tardiness risk value, the two values are assessed for the relationship between each other—namely, whether a total tardiness risk value is outside of a limit established by the threshold tardiness risk value. Most commonly, the evaluation involves determining whether the total tardiness risk value is a larger number than the threshold tardiness risk value. However, in embodiments, if the total tardiness risk value is calculated in a way such that a smaller number represents a greater likelihood of tardiness, than the evaluation involves determining whether the total tardiness risk value is a smaller number than the threshold tardiness risk value. As used herein, references to a total tardiness risk value being “larger” or “exceeding” (or the like) the threshold tardiness risk value includes both situations where a total tardiness risk value is a larger number than a threshold tardiness risk value, as well situations where the total tardiness risk value is a smaller number than the threshold tardiness risk value, if the total tardiness risk value is calculated in a way such that a smaller number represents a greater likelihood of tardiness.

In embodiments, a total tardiness risk value may be adjusted to be scaled to a selected threshold tardiness risk value. For example, a selected threshold tardiness risk value may be determined based on historical total tardiness risk values from two or more patients, where the total tardiness risk values for each patient were determined based on three different quantified tardiness risk values which were summed together to generate the total tardiness risk value. Continuing with the example, if for a patient of interest a total tardiness risk value is generated which is based on the summation of four different quantified tardiness risk values (rather than three), the total tardiness risk value for that patient of interest may not be appropriately scaled to the selected threshold tardiness risk value. Therefore, the total tardiness risk value for that patient of interest may be adjusted to scale to the selected threshold tardiness risk value. Specifically, in this example, since the total tardiness risk value for the patient of interest included 4 quantified tardiness risk values to yield the total tardiness risk value, as compared to the 3 quantified tardiness risk values which were used to generate the selected threshold tardiness risk value, the total tardiness risk value for the patient of interest may be reduced by 25% in order to adjust the total tardiness risk value for the patient of interest to the scale of the selected threshold tardiness value (i.e. to adjust 4 to the scale of 3). Any appropriate adjustment of a total tardiness risk value for a patient of interest in order to scale the total tardiness risk value to a selected threshold tardiness risk value may be used with systems and methods provided herein. Also, in embodiments, a total tardiness risk value for a patient of interest does not need to be adjusted to scale to a selected threshold tardiness value, even if the selected threshold tardiness value is based one or more total tardiness risk values obtained from a different number of quantified tardiness risk values than were combined to yield the total tardiness risk value for the patient of interest. For instance, certain methods of combining two or more quantified tardiness risk values into a total tardiness risk value may support the combination of different numbers of quantified tardiness risk values into a total tardiness risk value (e.g. with such a method, two different total tardiness risk values may be directly compared to each other, even if, for example, one was generated from three quantified tardiness risk values and the other was generated from four quantified tardiness risk values).

In embodiments, a total tardiness risk value may be generated by a computer program or software which, for example, correlates tardiness risk factors with quantified tardiness risk values or processes quantified tardiness risk values to determine a total tardiness risk value. In embodiments, a computer program may be run on a server of a health service center computer system. In embodiments, methods and steps provided herein may be computer-implemented and may be performed on a server having one or more processors. In embodiments, a processor may be configured to, for example, determine a total tardiness risk value and compare it to a threshold tardiness risk value. The processor, for example, may be part of a health service center computer system.

Taking Actions Upon Comparison of a Total Tardiness Risk Value to a Threshold Tardiness Risk Value

After the generation of a total tardiness risk value, in embodiments, an action may be taken. In embodiments, an action may be taken without comparing a total tardiness risk value to a threshold tardiness risk value. Such an action, for instance, may be proportional to the value of the total tardiness risk value.

In other embodiments, an action may be taken after comparing a total tardiness risk value to a threshold tardiness risk value. For example, upon the comparison of a total tardiness risk value to a threshold tardiness risk value, if the total tardiness risk value exceeds the threshold tardiness risk value, one or more actions may be taken. Actions may include, for example, contacting the patient to inquire whether he or she expects to be on time for the appointment, and if not, optionally whether he or she would like to reschedule the appointment (and if rescheduling the appointment, whether the patient has a desired date or time for the rescheduled appointment), automatically rescheduling the patient for a later appointment time, automatically canceling the patient's appointment, or scheduling a different patient at the patient's scheduled appointment time. In the event that a patient is automatically rescheduled for a new appointment time, the new appointment time may be based on the total tardiness risk value for the patient. For example, if a first patient's total tardiness risk value is much greater than a second patient's total tardiness risk value and both patients have their appointment rescheduled to a later time, the first patient's appointment may be rescheduled to a later time than the second patient's rescheduled appointment. In embodiments, if upon the comparison of a total tardiness risk value to a threshold tardiness risk value the total tardiness risk value does not exceed the threshold tardiness risk value, no action is taken.

In the event that a patient is automatically rescheduled for a new appointment time, the new appointment time may be based on the total tardiness risk value for the patient which is determined in units of time. For example, if a patient's total tardiness risk value is 14 minutes, the patient may be rescheduled for an appointment time at or around 14 minutes after the scheduled appointment time (e.g. at 10, 14, 15, or 20 minutes after the scheduled appointment time). In embodiments, if upon the comparison of a total tardiness risk value to a threshold value the total tardiness risk value does not exceed the threshold value, no action may be taken. This may be useful, for example, to avoid unnecessary changes when a patient is projected to only be slightly tardy. For instance, if a threshold tardiness risk value is 10 minutes and a patient's total tardiness risk value is 4 minutes, it may be most effective to not take any action (even though the patient is projected to be slightly late for the appointment). Since the patient is projected to be only slightly late to the appointment, it may be inefficient to reschedule the patient's appointment.

In embodiments, once a patient's total tardiness risk value is determined, an action may be taken without first comparing the total tardiness risk value to a threshold tardiness risk value. For example, the patient's appointment time may be automatically rescheduled for a new appointment time based on the total tardiness risk value for the patient, without comparing the total tardiness risk value to a threshold tardiness risk value. This may be useful, for example, in systems and methods where it is desirable to scheduled appointment times match patient arrival times as closely as possible.

In embodiments, a health service center computer system may contain instructions for determining the location of a patient having a scheduled appointment at a health service center. The location of the patient may be determined, for example, by obtaining information from a patient's user device which is capable of gathering geolocation data (e.g. a smartphone). Information regarding the location of the patient may be sent to, for example, a server of a health service center computer system of a system or method provided herein. In embodiments, when a patient is at or near to a health service center at which the patient has an appointment (e.g. within at least 1, 5, 10, 50, 100, 500, or 1000 meters of the health service center), one or more actions may occur. Such actions may include, for instance, any one or more of: automatically checking the patient in for his or her appointment, contacting the patient to remind the patient about the appointment and whether he or she would like to check in for the appointment, notifying the health service center that the patient is in the vicinity of the center, and adding the patient to a service queue in the health service center. In embodiments, based on geolocation data, a patient may be automatically checked in to his or her appointment upon entering the health service center where his or her appointment is scheduled. When a patient is checked in for an appointment and added to a queue to see a health care professional, the patient may also be provided with an estimated wait time until he or she will be seen by a health care professional.

In embodiments, systems and methods provided herein for appointment scheduling may take into account one or more factors which may influence the demand for appointments or the speed at which appointments may be performed at a health service center. For instance, systems and methods provided herein may obtain data regarding trends or levels of illness in the geographic area surrounding a health service center. Such information is available, for instance, from Google flu trend data or public health agencies. For example, if there is a comparatively high or increasing number of people in an area having or suspecting flu-like symptoms, a system or method provided herein may increase the number of available appointments, increase the number of available walk-in slots, or increase the number of health care providers at a health service center, in order to better meet the demands of the likely number of patients seeking treatment at the health service center. In another example, traditionally, health service centers may schedule appointments of patients at defined intervals—e.g. every 15 or 30 minutes. In contrast, a system or method provided herein may contain information regarding average amounts of time to perform one or more different medical-related procedures (e.g. a laboratory procedure). When scheduling patients for appointments at a health service center, systems and method provided herein may account for the average time to perform the medical-related procedure scheduled for one or more patients at one or more appointment times. Such average times may be generated, for example, from historical data for the performance of different procedures. Accordingly, systems and methods provided herein may create variable numbers of appointment slots in a given period of time, based at least in part on the estimated time required to perform one or more medical-related procedures scheduled in the same period of time. In addition, systems and methods provided herein may additionally or alternatively account for the average amount of time it takes a particular health care professional to perform a medical-related procedure scheduled for one or more patients at one or more appointment times (e.g. a particular nurse, doctor, or phlebotomist). Such average times may be generated from historical data for the time required by a particular health care professional to perform various medical-related procedures. For example, a first health care professional may typically take 5 minutes to perform an immunization, whereas a second health care professional may typically take 8 minutes to perform the same immunization. Accordingly, systems and methods provided herein may create variable numbers of appointment slots in a given period of time, based at least in part on the estimated time required by a particular health care professional to perform one or more medical-related procedures scheduled in the same period of time.

Systems and methods provided herein may further include permitting a patient to make an appointment based on the patient's intended arrival time for the appointment at a health service center, rather than based on a time that a patient is required to arrive by. For instance, traditionally, if a patient makes an appointment for a particular time, he or she is required to arrive at the appointment by that particular time, or risks a negative consequence, such as losing the appointment, having the appointment rescheduled to a different day, or paying a financial penalty. In contrast, with systems and methods provided herein, a patient may make an intent-based appointment for a particular time. With an intent-based appointment time, a patient may be expected to arrive within a certain window of time around particular time, rather than necessarily at a precise time. For example, with an intent-based appointment, a subject may make an appointment for a given time (e.g. 2 PM), and may be expected to arrive within 5, 10, 15, 20, 30, 45, 60, 90, 120, 150, or 180 minutes before or after the given time without negative consequences for the patient (e.g. no financial penalty or rescheduling of the appointment to a different day). Other examples of intent-based appointment scheduling may include, for example, an intended appointment for a particular time of day (e.g. morning, afternoon, or evening), or for a window of time (e.g. between 1 PM and 4 PM or between 7 AM and 12 PM). In embodiments, to facilitate intent-based appointment scheduling, systems and methods provided herein may analyze one or more factors such as the location of the subject or the status of local public transportation systems, and based on the analysis, optionally automatically take an action if it is predicted that the patient will be late for his or her appointment, such as to reschedule the patient for a later time or to accept a new patient at or around the time of the original patient's appointment. In addition, systems and methods provided herein may additionally or alternatively take into account one or more factors which may influence the demand for appointments or the speed at which appointments may be performed at a health service center, as described above. For example, a health service center may be staffed with an appropriate number of health care professionals to meet the expected demand of patients during a given window of time (e.g. more health care professionals may be needed during a Wednesday afternoon in a period of high flu incidence than during a Saturday morning in a period of low flu incidence). Health service centers which are staffed with an appropriate number of health care professionals to meet the expected demand of patients during a given window of time may further be well-suited to accommodate patients who make appointments with an intent-based appointment time, rather than a strict precise appointment time.

As used herein a “health service center computer system” refers to a computer system which is operatively connected to a health service center. A health service center computer system may include software and hardware for storing information or performing computing operations relating to the health service center. In embodiments, a health service center computer system may perform mostly or exclusively operations relating to appointment scheduling and check-in. In other embodiments, a health service center computer system may perform some or many other operations besides appointment scheduling and check-in. A health service center computer system may, for example, store patient information such as home addresses, contact information (e.g. phone number, e-mail address), billing information, emergency contact information, insurance information, appointment histories, medical records, user login names and passwords, and healthcare provider, and it may provide operations such providing information to a user, or permitting a user to request, change, check-in to, or cancel appointments or request information from a health service provider. In embodiments, a health service center computer system may be limited to a device or network specific to a particular health service center where a patient is to receive a service. In other embodiments, a health service center computer system may be operably connected to a wide-area network (e.g. the Internet), such that the health service center computer system may be accessible from any appropriate computing device connected to the wide-area network. In embodiments, a health service center computer system may contain components which are stored locally at a particular health service center location (e.g. a computer at a health service center), as well as components which are not at a particular health service center location (e.g. a server located at a different location from the health service center, which is accessible in over the Internet). A health service center computer system may contain a distributed network of one or more servers, data storage units, or processors. Any of the methods described herein, such as for performing appointment scheduling, rescheduling, check-in and determination of tardiness risk values or total quantified estimated tardiness amounts may be performed on or with the aid of the health service center computer system. In embodiments, one or more computers at a health service center may be operatively connected to a health service center computer system through a network (e.g. the Internet). In embodiments, multiple different health service centers (i.e. at different locations) may be operatively connected to a common health service center computer system.

As used herein, a “computing device” refers to any device which may collect, display, store, or process digital data, and may include, for example, servers, personal computers, data storage units, hard drives, portable digital media, smartphones, computer systems, mobile devices, external data sources, and user devices. In embodiments, systems or elements thereof described herein may be implemented as distributed network of devices and components connected via a network.

In embodiments, a health service center computer system may contain one or more servers. Turning to FIG. 1B, in the event a health service center computer system 170 contains two or more servers, in embodiments, at least one server may be a primary server 110 and at least one server may be a caching server 120. A primary server 110 may contain, for example, a processor 111, a memory unit 112, and a data storage unit 113. The processor 111 of a server may be a hardware structure which performs computational operations of a computer program. In embodiments, a processor 111 may carry out instructions stored in a tangible computer readable medium. The processor 111 may contain one or more microprocessors. A memory unit 112 is a structure for storage of digital information which typically uses volatile storage and is rapidly or directly accessible by a processor (e.g. random access memory (RAM)). A data storage unit 113 is a structure for storage of digital information which typically uses non-volatile storage, and which typically has a much larger storage capacity than a memory unit 112, but is less quickly accessible by the processor 111 than the memory unit (e.g. hard drive). In embodiments, the memory unit 112 or data storage unit 113 may store non-transitory computer readable media, which may include, for example, code, logic, or instructions for performing methods provided herein. A primary server 110 may have any number of processors 111, memory units, 112, or data storage units 113 (e.g. 1, 2, 3, 4, 5, 6, 7, 8, 9, 10, 15, 20, 25, 50, 100, 1000, or more of any or each of the processors, memory units, or data storage units). A primary server 110 may also contain other components, such as a removable media drive (which may accept, for example, CDs, DVDs, floppy disks, or magnetic tape-based storage), input-output (I/O) channels, buses, network interfaces (wired or wireless structures for facilitating data transfer between a server a network), or power supplies. A primary server 110 may have a variety of different shapes and structures. For example, a primary server 110 may be a dedicated server, or it may be part of a computer which contains other features (e.g. a monitor, peripherals, etc.). In some embodiments, the primary server 110 may be part of, for example, a personal computer or a smart phone.

A system provided herein may contain non-transitory tangible computer readable media. Computer readable media can be any available media which can be directly or indirectly accessed by a processor or server of a system provided herein. Computer readable media may include volatile and nonvolatile media, as well as removable and non-removable media. Computer readable media may be implemented in any method or technology for storage of information, such as computer readable instructions, data structures, program modules, or other data. Computer storage media may include, but is not limited to, RAM, ROM, EEPROM, flash memory or other memory technology, CD-ROM, digital versatile disks (DVDs) or other optical disk storage, magnetic cassettes, magnetic tape, magnetic disk storage or other magnetic storage device, or any other medium which can be used to store information and which may be accessed by a processor or server.

A server of a health service center computer system may be operably connected to one or more external data source 140 (e.g. a website with information of interest, a GPS associated with a computing device of interest, a different server, a hard drive); the server may obtain information from such sources as-needed or at regular intervals. In embodiments, a server may include data mining hardware or software, such as software configured to search the Internet or predetermined web sites (e.g., “weather.com”) on the internet to obtain data of interest. In embodiments, an external data source 140 may be a data storage unit operably connected to the server. A server may have load balancing, task management, and backup capacities. The components of a server may be within a single housing unit, or they may be distributed between two or more housing units. A server may be implemented as a distributed network of processors, memory, and storage units. A server may contain or be operably connected to a database (for example, the database may be in a data storage unit of the server or in an external data source). The processor of a server may run a computer program or software, the instructions of which may be provided from, for example, a data storage unit, removable media, or a data storage unit operably connected to the server. In embodiments, two or more servers may act together to function as a server. Servers may communicate with any number and type of computing devices. The server may engage in one-way or two-way communication with computing devices. Other server components or configurations not explicitly discussed herein but known in the art may be included in servers and systems described herein.

A server of a health service center computer system may contain or be operably connected one or more databases of information relevant to appointment scheduling or check-in. For example, databases may contain information relating to user or patient accounts, such as patient home addresses, patient contact information (e.g. phone number, e-mail address), billing information, emergency contact information, insurance information, appointment histories, medical records, user login names and passwords, patient healthcare provider information, or other information. In other examples, databases may contain information relating to appointment scheduling or to historical patient information which may correlate various tardiness risk factors with quantified tardiness risk probabilities or quantified estimated tardiness amounts. A server may, with the aid of a processor, use data from one or more sources to perform methods relating to appointment scheduling and check-in. For example, a server may use data from a data storage unit within the server or from an external data source in order to perform one or more operations or analyses.

Referring again to FIG. 1B, a caching server 120 may have any of the components or configurations of the primary server 110 described elsewhere herein. Generally, the caching server 120 is optimized for temporary storage of frequently-accessed content from the primary server 110, in order to increase the speed at which the content can be delivered to a client/user and to decrease the number of operations required to be performed by the primary server 110 (and in turn, to increase the performance of the primary server 110). The caching server 120 may store content in either or both of the memory unit 122 or data storage unit 123. In systems and methods provided herein, the caching server may store, for example, appointment information for one or more health service centers. The caching server 120 may be configured to regularly update from the primary server 110 its cached content. In embodiments, a caching server 120 may be located in a particular geographic area, and may be configured to respond to data requests from users the same or related geographic areas. For example, a first caching server 120 could be provided in North Carolina to respond to requests based in the eastern United States, a second caching server 120 could be provided in Texas to respond to requests based in the central United States, and a third caching server 120 could be provided in California to respond to requests based in the central United States. In embodiments, two or more caching servers 120 may be operably connected to a single primary server 110. In other embodiments, two or more caching servers 120 may be operably connected to two or more primary servers 110. In embodiments, a system provided herein may contain more caching servers 120 than primary servers 110, more primary servers 110 than caching servers 120, or equal numbers of primary servers 110 and caching servers 120.

In embodiments, a health service center computer system may contain any number of servers. In embodiments, all, some, or none of the servers may be differentiated between primary servers and caching servers.

Systems and methods provided herein may involve data transfer over a network. In embodiments, components of systems provided herein may be operatively connected via a network. The network may be any structure which can support the operable connection of and data transfer between two or more computing devices, such as a local area network (LAN) or a wide area network (WAN), and may include, for example, an intranet, an enterprise private network, the Internet, cellular, or satellite networks. The network may include, for example, one or more of wireless connections, wired connections, or fiber optic connections. Computing devices (e.g. servers, external data sources, and user devices) may connect to the network by wired or wireless technologies. For example, a computing device may connect to the network via wired technologies such as a dial-up connection with a modem, a direct link such as TI, ISDN, cable, Firewire, USB, or Ethernet wire. In other examples, a computing device may connect to the network via wireless technologies such as Bluetooth, RTM, infrared (IR), radio frequency (RF), ZigBee, Z-wave, wireless USB, code division multiple access (CDMA) or global system for mobile communications (GSM). In embodiments, data may be encrypted before it is transmitted over the network.

As used herein, an “external data source” may be any computing device which may store, transmit, receive, or gather data that may be accessed by or sent to a health service center computer system (e.g. to a server of a health service center computer system). External data sources include, for example, servers, hard drives (e.g. IDE, ATA, or SATA drives), databases, personal computers, data storage units, hard drives, portable digital media, smartphones (e.g. Apple iPhone, Android-enabled phone), mobile devices, and computer systems, global positioning system (GPS) devices. An external data source may be portable or non-portable/at a fixed location. In embodiments, an external data source may be capable of obtaining geolocation data, such as by wireless triangulation or a GPS system. In embodiments, an external data source may be on or associated with a subject (e.g. on a subject's wrist or in a subject's pocket or handbag). The external data source may be a portable computing device in proximity to the subject such that the measured location of the device corresponds to the location of the subject. The external data source may be a portable computing device the subject carries for other purposes. For example, the external data source may be a smart phone, such as an iPhone or Android-enabled phone, capable of gathering geolocation data, such as with the aid of a GPS module of the device. In embodiments, an external data source may be an iPad or other portable computing device, such as a watch capable of gathering geolocation data. A client-server relationship, peer-to-peer, or a distributed relationship, may be provided between the an external data source and the health service center computer system. In embodiments, an external data source may communicate directly or indirectly with a health service center computer system. For example, an external data source may have a direct wired linkage to a server of a health service center computer system. In another example, an external data source may communicate wirelessly with a server of a health service center computer system. In another example, an external data source may communicate with a server of a health service center computer system when the external data source is connected to a personal computer via a wire, and when the personal computer is connected to the Internet (which may be operably connected to the health service center computer system). In embodiments, an external data source may be operatively coupled to a primary server of a health service center computer system. The external data source may be coupled to a primary server such that data travelling between the external data source and the primary server passes through a caching server as it travels between the external data source and the primary server. In other embodiments, an external data source may be coupled to a primary server such that data travelling between the external data source and the primary server does not pass through the caching server as it travels between the external data source and the primary server. In embodiments, a system provided herein may be configured such that an external data source is operatively coupled to a primary server of a health service center computer system without passing through or involving a caching server. With systems and methods provided herein, any number of external data sources may be in communication with a server, such as 1, 2, 3, 4, 5, 10, 15, 20, 25, 100, 1000 or more external data sources may be in communication with a server.

As used herein, a “user device” may be any computing device with which a user may review, input, or change appointment-related information. User devices may include, for example, personal computers, tablet computers, smartphones (e.g. Apple iPhone, Android-enabled phone), mobile devices, or computer systems. A user device may be portable or non-portable/at a fixed location. A user device may contain one or more user interfaces. User interfaces may provide information to a user, obtain inputs from a user, or both. A user interface may have a display, such as a cathode ray tube, plasma, liquid crystal display (LCD), or light-emitting diode (LED)-based display. In embodiments, a user interface may include a graphical user interface (GUI) configured to display information to a user on a display, such as appointment time and availability information. A GUI may also be configured to receive information from a user, such as by capacitive or resistive touch-screen functions. In some situations, user interfaces may include camera for video or still images, a microphone for capturing audible information (e.g., a subject's voice), speakers for providing audible information, a printer for printing information, or a projector for displaying images and/or video on a predetermined viewing surface. Other user interfaces of a user device may include, for example, a keyboard, touch pad, or a computer mouse. A user device may contain a processor and local memory and data storage. In embodiments, a user device may be specialized for use by employees of a health service center at the health service center. Such a user device may also be referred to as a “health service center terminal”. Employees or others at a health service center may use a health service center terminal to, for example, enter or obtain patient information (e.g. patient location, patient expected arrival time, patient scheduled appointment time, patient scheduled laboratory procedure(s)). As used herein, a “user” may include any individual who directly or indirectly interacts with a health service center computer system, and may include, for example, a patient who has an appointment at a health service center. In embodiments, a user may be a person other than a patient who has an appointment at a health service center (e.g. a user may be an employee at a health service center who wishes to access information about the patient or an assistant of the patient who wishes to assist the patient with entering information into or obtaining information from the health service center computer system).

In embodiments, certain computing devices may function as both an external data source and a user device for systems and methods provided herein. For example, a GPS receiver-containing tablet computer may both: i) obtain patient location data which is provided to a server of a health service center computer system running a software program described herein (and thus, it may function as an external data source), and ii) provide a user interface such as a touch screen which may display and receive user input regarding appointment times (and thus, it may function as a user device). In other embodiments, certain computing devices function as either an external data source or a user device. For example, a stand-alone hard drive operatively coupled to a server of a health service center computer system is an external data source but not a user device. In another example, a device having just a display which is located at a health service center to display appointment time information for patients may function as a user device but not an external data source.

In embodiments, a user may be able to interact with software of a health service center computer system through a client application running on a user device. A client application may be, for example, a World Wide Web (WWW)-based interface. A WWW-based interface may be provided, for example, at a specific URL (e.g. a web page), which users may access via the network through a user device. A user may request a WWW-based interface at a specific URL, and the content may be delivered to the user device from, for example, a server of the health service center computer system. In embodiments, users may input information on a WWW-based interface, and the information may be provided to a server of a health service center computer system. In embodiments, a WWW-based interface may permit a user to log in to a user account, to permit the user to access one or more interconnected web pages (e.g. web pages associated with the user account). In embodiments, a WWW-based interface may provide appointment-related information. With the WWW-based interface, a user may optionally be able to, for example, view appointment-related information, request an appointment, change an appointment date/time, cancel an appointment, or provide special instructions or comments relating to a past or upcoming appointment.

In addition to the system components and configurations described above and elsewhere herein, it is also noted that other suitable system components and configurations may be used with systems and methods provided herein. For example, embodiments of methods provided herein can be implemented with a computer system that includes a back-end component (e.g. a server) and a front-end component (e.g. a user computer having a GUI or Web browser through which a user can interact with a computer software for performing methods provided herein), in which the back-end component and front-end component are interconnected by any combination of hardware or software for digital data communication. In other examples, embodiments of methods provided herein can be implemented using a single computing device (e.g. where the computing device stores relevant data, contains one or more processors for performing operations described herein, receives user information, and displays information to a user). Also, it is noted that the relationship between objects depicted in FIGS. 1A and 1B and elsewhere herein is exemplary, and other relationships are within the scope of systems and methods provided herein. For example, although FIG. 1 depicts an external data source being connected to a health service center computer system via a network, in embodiments, an external data source may directly link to a component of a health service center computer system (e.g. a server), without connecting through a network. In another example, a system provided herein may contain only a primary server, rather than a primary server and a caching server, wherein the primary server fulfills the function of both the primary server and caching server. In another example, systems provided herein may contain external data sources but not user devices, or may contain user devices but not external data sources. In another example, a server of a system provided herein may contain one or more computer memory devices, rather than necessarily containing both a distinct data storage unit and memory unit.

In embodiments, provided herein are systems and methods for facilitating patient check-in at a health service center. In embodiments, systems and methods for patient check-in may involve near-field communication (NFC) technologies. NFC involves transmission of information through very short-range radio, typically 10 centimeters or less. To transmit information by NFC, two NFC chips are brought into close proximity, upon which information may be transferred between the chips (the chips may be capable of one or two-way communication). An NFC chip may be part of a powered, NFC-enabled device. Such powered NFC chips may generate a radio frequency field. The radio frequency field generated by a powered NFC chip may power an unpowered, “passive” NFC chip. Passive NFC chips may be referred to herein as NFC “tags”, and such NFC tags may have a very small size and may be incorporated into various forms, shapes, and structures.

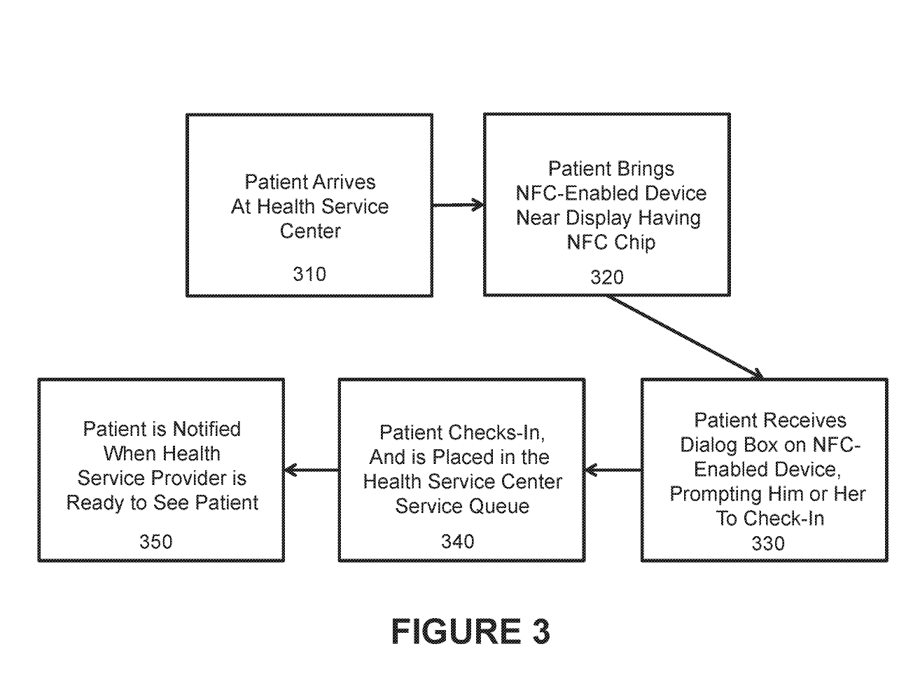

FIG. 3 provides an exemplary schematic of a check-in procedure according embodiments of systems and methods provided herein. Turning now to FIG. 3, a patient may arrive at a health service center 310. The patient may have a portable NFC-enabled device in his or her possession, such as an NFC-enabled phone, watch, or computing device. At one or more locations within the health service center, a display containing an NFC chip may be present. The NFC chip may be a passive NFC tag. The display may take any form, such as a poster, banner, card, or other configuration. The display may be labeled with a message or symbol denoting the location of a NFC chip in the display. The display may inform the patient to swipe the NFC chip in the display in order to perform one or more operations, such as to check-in for an existing appointment, to create a request for a new appointment, to create an account with the health service center, or to request to consult with a health care provider.

The patient may bring his or her NFC-enabled device near the NFC chip in the display (i.e. swipe/tap their device at the chip) 320, so that the NFC-enabled device can receive information from the NFC chip in the display. Typically, in order to successfully receive information from the NFC chip, the patient may bring their NFC-enabled device within 1 cm to 5 cm of the NFC chip, although other distances are not excluded. Prior to swiping his or her NFC-enabled device near the NFC chip in the display, a patient may adjust settings in the NFC-enabled device in order to turn on NFC functionality in the device. The NFC chip in the display may provide information to the NFC chip in the NFC-enabled device in a standard data format, such as NFC Data Exchange Format (NDEF). In embodiments, custom Multipurpose Internet Mail Extensions (MIME) type may be used.

After a patient swipes his or her NFC-enabled device at the NFC chip in the display, his or her NFC-enabled device may receive and display information from the NFC chip. In embodiments, information from the NFC chip may prompt a dialog box to open on a screen of the NFC-enabled device. The dialog box may provide various different information or instructions to a patient. The dialog box may, for example, prompt a patient to log into an application associated with a health service center computer system.

In embodiments, a patient may be able to obtain a software application which can operate on a NFC-enabled device, and permit the patient to access the health service center computer system from an NFC-enabled device. For example, a patient may download the application from the Internet, or the application may be pre-installed on a NFC-enabled device the patient acquires.

In embodiments, the NFC chip in a display in a health service center may contain information relevant to the specific health service center in which the NFC is located (e.g. the name or ID of the service center, the services offered at the service center, or a location-specific service queue).

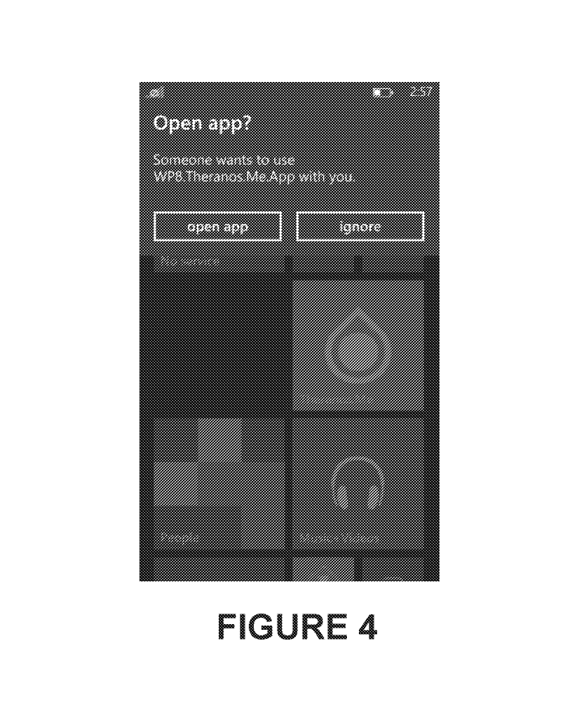

In an example, if a patient is not logged into the health service center computer system at the time of swiping his or her NFC-enabled device at the NFC chip in the display, the patient may be presented with a dialog box which prompts the patient to log into the application. As used herein, a reference herein to logging into (or the like) a health service center computer system includes the action of logging into a software application operated by a health service center computer system. A screen shot of an exemplary dialog box which prompts a patient to log into a health center computer system is provided in FIG. 4. In embodiments, a dialog box may provide a patient with the opportunity to create an account with the health service center computer system, if the patient does not have an account. Upon logging into the health service center computer system, the patient may be provided with the opportunity to check-in to an appointment or to perform other operations, as described further herein.

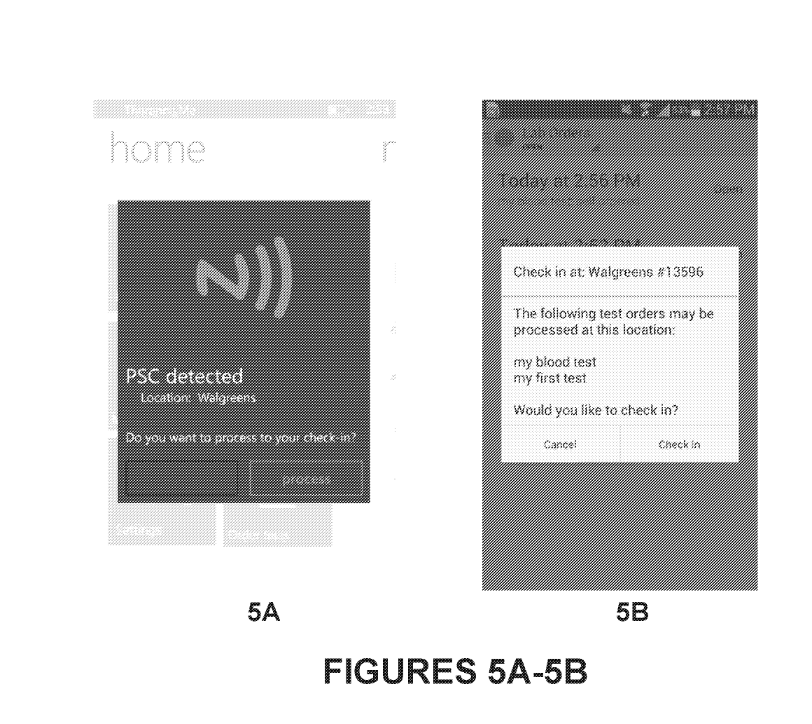

If a patient is logged into the health service center computer system at the time of swiping his or her NFC-enabled device at the NFC chip in the display, the patient may be presented with a dialog box which may contain personalized information or instructions. For example, if the patient has an existing appointment at the health service center close to the day or time of swiping his or her NFC-enabled device at the NFC chip in the display, the dialog box may prompt him or her to check-in for the appointment 330. Screen shots of exemplary dialog boxes which may provide a patient with an opportunity to check in for an appointment are provided in FIGS. 5A (Windows™-based phone) and 5B (Android™-based phone). In another example, if a patient does not have an existing appointment at the health service center at the time of swiping his or her NFC-enabled device at the NFC chip in the display, the dialog box may provide the patient with instructions or procedures for making an appointment. For example, if the patient has an existing appointment at the health service center relatively far from the day and/or time of swiping their NFC-enabled device at the NFC chip in the display (e.g. 24 hours or more), the dialog box may provide the patient with options for canceling or rescheduling the appointment. It should be understood that the dialog box may provide or may link to more than one option for actions (e.g. it may provide the option of checking in to the appointment, to change the appointment time, to cancel the appointment, etc.).

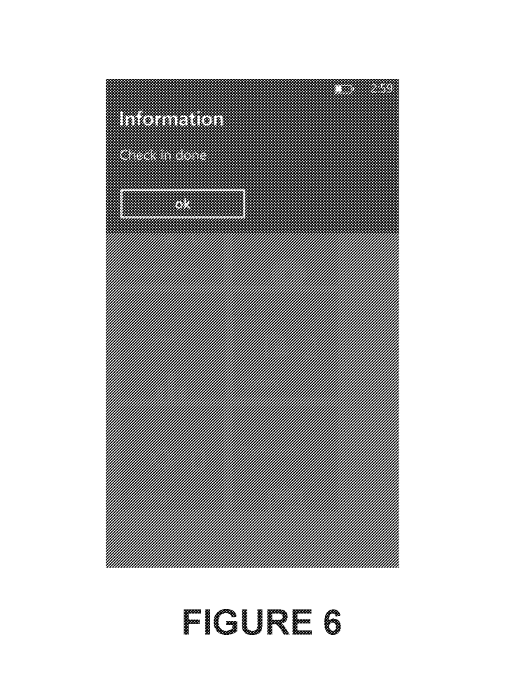

When a patient has checked in for an appointment, a dialog box may be provided on the NFC-enabled device indicating that the patient's check-in was successful. An exemplary screen shot of a dialog box showing a patient that he or she has successfully checked in to an appointment is shown in FIG. 6.

Upon checking-in for an appointment through the NFC-enabled device, information regarding the patient (e.g. name, patient number, etc.) may be put into a service queue at the health service center 340. The service queue may be patient-specific. For example, a patient may have an appointment with a specific health care provider at a specific time, and the service queue may simply inform the specific health care provider that the patient has checked-in at the health service center. Alternatively, the service queue may accommodate multiple patients. For example, the patient may have an appointment for a procedure which may be performed by any one of various health care providers present at the health service center, and the service queue may include multiple patients with appointments for similar procedures. In this example, patients may be added to the service queue sequentially upon check-in, and may be seen in their order of check-in by the next available health care provider at the health service center. In embodiments, a patient may be added to a service queue without having had an appointment prior to arriving at the health service center, if the patient requested a service after arriving at the health service center.

After being added to the service queue, the patient may be informed when a health care provider is ready to see the patient 350. The patient may be informed by any appropriate method. For example, the patient may receive a notification (e.g. a text or e-mail) on a computing device (e.g. a NFC-enabled phone), one or more electronic screens (e.g. LCD, plasma, etc.) in the health service center may display the information, or the information may be provided audibly in the health service center (e.g. over a loudspeaker). In embodiments, a patient may be informed that a health care provider is ready to see the patient by multiple methods.

In certain embodiments of systems and methods provided herein, one or more of the following events may occur when a user swipes a NFC-enabled device at NFC chip in a display. In embodiments, one or more of the following events may occur with Windows™ based systems. An NFC chip in a display (e.g. a NFC tag) may contain application identification (ID) information for a software application of a health service center computer system. When the NFC-enabled device obtains the application ID from the NFC tag, if the NFC-enabled device already contains the application, it may show a dialog box on its screen prompting the user to open the application. Alternatively, if the NFC-enabled device does not contain the application, it may show a dialog box directing the user to download the application from an appropriate location (e.g. an online “app” store). Once the user opens the application (or starts the process of opening the application), additional information may be read by the NFC-enabled device from the NFC tag in the display. The additional information may, for example, instruct the application how to handle certain information from the NFC tag in the display. For example, the NFC tag may use a string with a certain code in order to instruct the application that certain parameters provided from the NFC tag should be used for checking the user into an appointment. Information that may be provided from the NFC tag may include information which is used to check the user in to an appointment. For example, this may include an ID code of the particular health service center and a name of the particular health service center. The application may take this information, as well as information regarding the user (e.g. a User ID), to, for example, a server, which may process the user information and add the user to a service queue at the health service center. The server, for example, may be part of the health service center computer system.

In certain embodiments of systems and methods provided herein, one or more of the following events may occur when a user swipes a NFC-enabled device at NFC chip in a display. In embodiments, one or more of the following events may occur with Android™ based systems. An NFC chip in a display (e.g. a NFC tag) may contain an ID code of the particular health service center and a name of the particular health service center. The first or other NDEF record sent from the NFC tag may contain this information. An application operating on the NFC-enabled device for the computer system of the health service center may receive this information from the NFC chip, may display the name of the store on the NFC-enabled device, and may pass this information in combination with user ID information to a server, in order to add the user to the service queue at the health service center. A second NDEF record that may be sent from the NFC tag may be a link to a location where the application for the health service center computer system may be obtained by a user (e.g. the Google Play store), for in the event the user does not have the application for the health service center computer system.

With systems and methods involving NFC technologies provided herein, a user may be able to perform various operations at a health service center without needing to speak with a live human representative. For example, with systems and methods provided herein, a user may request an appointment, check in to an appointment, cancel an appointment, change an appointment, or make a payment at a health service center without needing to speak with live human representative. Nonetheless, in embodiments, a human representative may be available at a health service center to speak with a user, if the user so desires. By providing users with the option of performing various tasks at a health service center without needing to speak with a human representative, the convenience or confidentiality of the process for the user may be increased.

In further embodiments of systems and methods provided herein, a patient may have the option of performing an early check in for an appointment at a health service center. The early check in may be performed at the time of scheduling the appointment, or at a point prior to arriving for a scheduled appointment at the health service center. In embodiments, an early check in may be performed at least 1 hour, 2 hours, 4 hours, 8 hours, 12 hours, 1 day, 2 days, 3 days, 7 days, 14 days, or 30 days before a scheduled appointment. When checking in early for the appointment, the patient may have the option of providing one or more types of information to a health service center computer system to facilitate one or more steps related to the patient's appointment. The patient may have the option of providing, for example, his or her: insurance information, lab or medical procedure order, signed consent form, guardian, next of kin, or emergency contact information, home address, contact information, billing information, or medical history. In embodiments, a patient may have the option of providing information to a health service center computer system by obtaining an image of a document (e.g. by scanning, with a camera, or other data acquisition technology that may be developed in the future), and electronically transmitting the document to the health service center computer system. This process may be facilitated by a patient's use of an app from the health service center computer system, which a patient may use on a user device. For example, a patient may take an image of a sheet of paper containing a doctor's order for a laboratory test with a camera on his or her smart phone, and then upload the image to the health service center computer system via an app on his or her smart phone. In embodiments, the health service center computer system may support text or image recognition, such that the content of documents for which images are obtained may be determined. In embodiments, during an early check in procedure, a patient may provide to the health service center computer system both a i) his or her insurance information, and ii) information regarding the health-related procedure(s) (e.g. laboratory test) ordered by a health care professional for the patient. For example, a patient may take an image of his or her insurance card and an image of his or her lab order form, and upload the images to the health service center computer system. Upon the receipt of the patient's insurance and health-related procedure information, a health service center computer system may determine the amount that the patient will have to pay out of pocket for the health-related procedure(s), and provide this information to the patient (e.g. on a user device). The amount that that the patient will have to pay for the health-related procedure(s) may be for any reason—e.g. it may be the amount that is the patient's co-pay with his or her insurance plan or it may be the entire cost of the procedure, if the patient's insurance plan does not cover the procedure or if the patient does not have insurance. In some embodiments, a patient will be informed of the amount he or she will have to pay, regardless of the amount. In some embodiments, a patient may only be informed of the amount he or she will have to pay out of pocket if the amount exceeds a certain amount. In some embodiments, a patient may only be informed of the amount he or she will have to pay out of pocket if the amount is less than a certain amount. The certain amount value may be set by the health service center computer system or the patient. These steps may provide a patient with clearer information regarding the cost of a health-related procedure and may facilitate planning for a procedure by a patient. Optionally, the patient may be informed that he or she qualifies for coverage but will also be required to make a copayment, wherein the message does not set forth the exact numeric amount of the co-pay. In this manner, the subject may be referred to their insurance card for the exact amount of the co-pay. Optionally, some embodiments may present a confidence score associated with a co-pay determination, wherein the confidence score may relate to the accuracy of the determination. In embodiments, any of the steps described herein as early check in steps may also be performed at a health service center at the time of an appointment, if a patient checks in at a health service center for an appointment. In embodiments in which a system or method provided herein provides a patient with the amount that the patient will have to pay out of pocket for a health-related procedure prior to the performance of the procedure, the health service center computer system may contain or have access to information regarding different insurance plans' coverage of different health-related procedures. For example, the health service center computer system may contain or have access to information regarding whether a particular insurance plan covers a certain procedure or whether a particular insurance plan requires a co-payment from a patient for a certain procedure, and if so, the amount of the co-payment.

Optionally, some embodiments related to the comparison of insurance coverage to desired testing may involve a data capture step for patient information, health-related procedure information, or insurance information. Optionally, this data capture step can occur on the insurance carrier's servers, a health service center computer system server, or another party's server. Optionally, the determination of insurance coverage can be made using the same server(s) that performs the data capture step. Optionally, the determination of insurance coverage can be made using server(s) different from those that perform the data capture step. In some embodiments, wherein the data capture is below a quality threshold, additional secondary or tertiary information can be used to improve the accuracy of data capture. By ways non-limiting example, if the laboratory order form is scanned or photographed by a patient and uploaded to a health service center computer system, and the form is then recognized through pattern or other data matching to be one from a group of laboratory forms known by a software program of a server, the known form(s) can be used to verify or fill-in information such as test name, type of test, or other information imperfectly recorded during data capture (e.g. if the patient takes a low-quality picture of the form, or the form is partially damaged). Optionally, some embodiments may be configured to alert a human or other resource to assist in verifying the captured data. Although this section is discussed in the context of health-related procedure information, this information may also be applicable to the captured data relating to other types of information such as but not limited to patient information or insurance information. Optionally, at least some embodiments herein may also have an additional step related to data capture wherein if the quality of image or other data capture is below a pre-determined level or other threshold, the user can be requested to recapture the image or data. Optionally, this request can be made by a user device that makes the initial image or data capture or it can be a request that comes from a server that is processing the data. Feature related to quality may include but are not limited to image contrast, image size, image focus, partial image capture, or other image errors.

In embodiments, the results of a health-related procedure, which are associated with an appointment or patient check-in performed according to a method disclosed herein may be communicated to the patient by a wireless link, such as, e.g., to a cell-phone, a computer connected to WiFi, or other user device connected to a network via a wireless link. In embodiments, such wireless communication of results of a health-related procedure, may comprise a text message; may comprise a voice message; may comprise a real-time voice conversation; may comprise information in an app from a health service center computer system; or combinations thereof. In embodiments, the results of a health-related procedure associated with an appointment or patient check-in performed according to the methods disclosed herein may be communicated to the patient by email. In embodiments, the results of a health-related procedure associated with an appointment or patient check-in performed according to the methods disclosed herein may be communicated to the patient by physical mail.

With systems and methods provided herein, a user may be able to perform various operations at any location (e.g., at a health service center, or at any other location), with or without the need to speak with a live human representative. For example, any of the steps described herein as early check in steps may be performed at any location (e.g., at a health service center, or at any other location), with or without the need to speak with a live human representative. In embodiments, one or more step, action, or service may be performed by, or may be performed with the assistance or participation, of a live human.

In embodiments, a step associated with making an appointment, or with patient check-in, or both, may be a step of determining the amount of a co-payment required for a service or for services to be performed prior to, during, or after the appointment, or pursuant to the appointment. A step of determining the amount of a co-payment may include determining the amount of a copayment for a service; may include determining the amount of a copayment for a service for a group or class of patients; may include determining the amount of a copayment for a service for a particular patient; may include determining the amount of a copayment for a service for a patient having a particular condition; and may include communicating a copayment amount to a patient, or patient representative.

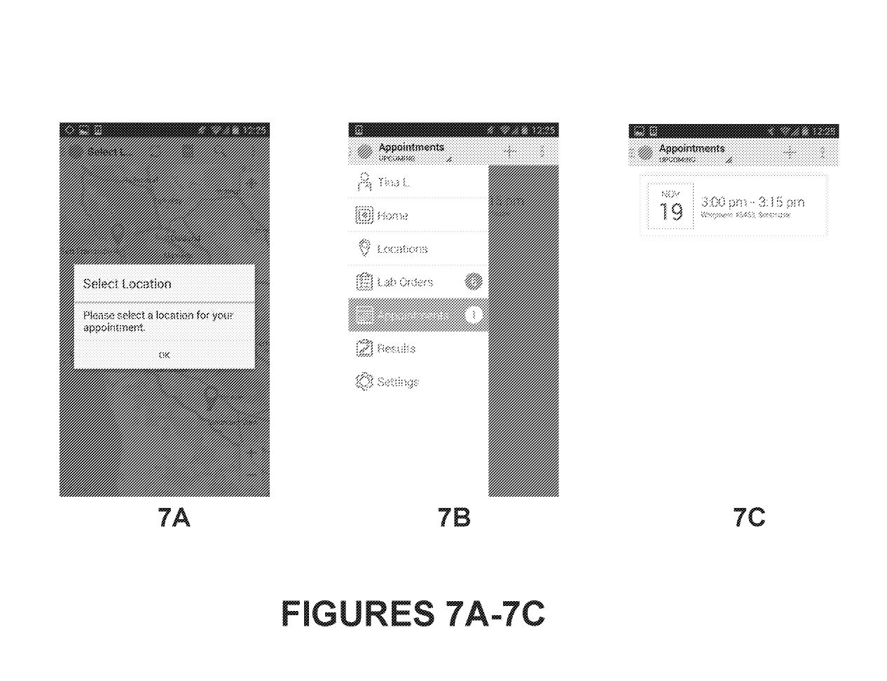

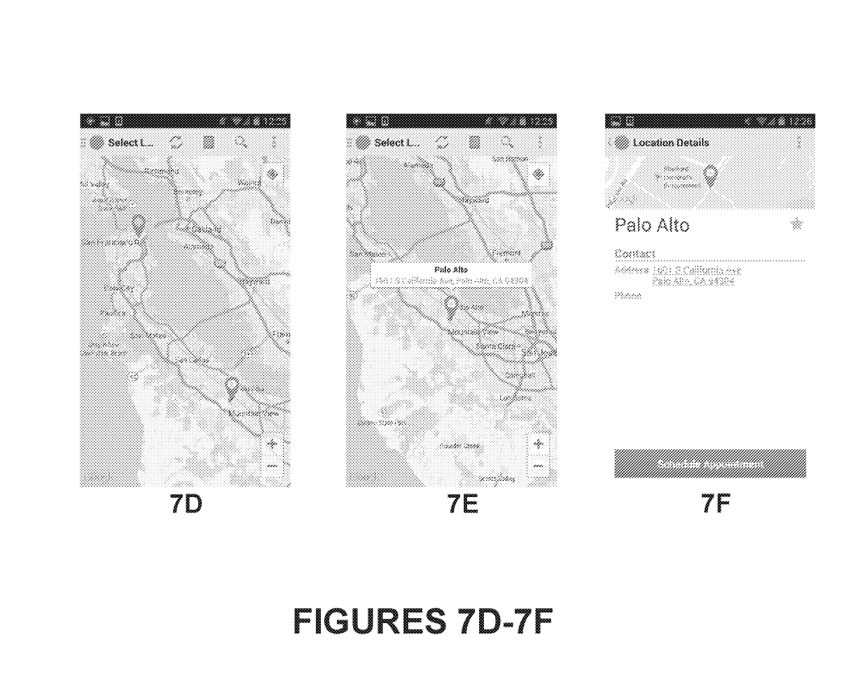

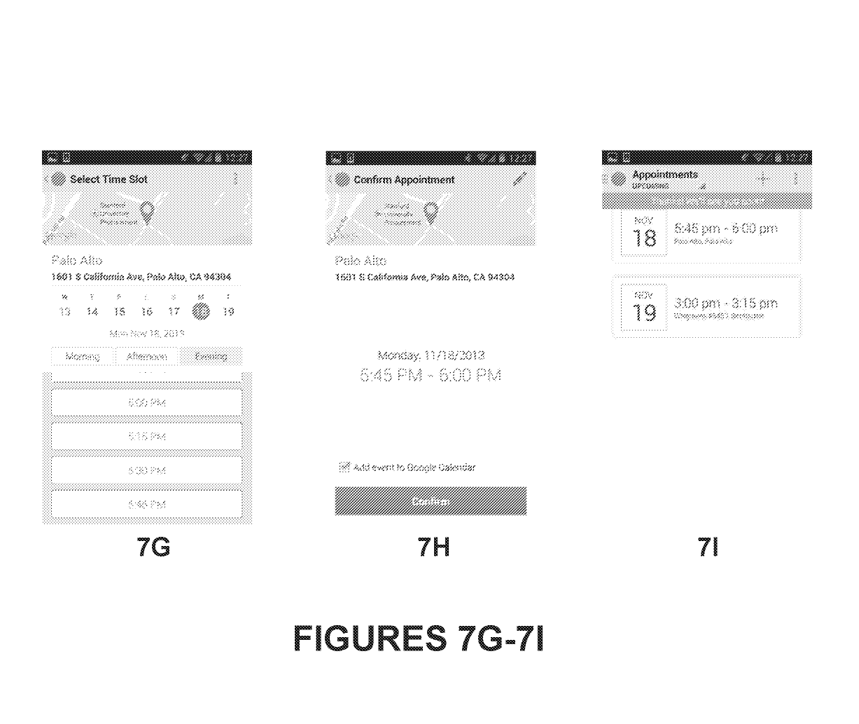

In embodiments, one or more steps of methods for appointment scheduling or check in provided herein may be performed with reference to FIG. 7. A user may access an application of a health service center computer system on a user device, such as a smart phone or tablet computer. An application of a health service center computer system operating on a user device may provide various different functionalities to a user. In embodiments, an application may permit a patient to schedule an appointment for a service at a health service center. For example, a patient may access an appointment-scheduling capability in the application from a feature of the application, such as a page which lets a user select an appointment location from a map-based selection feature (FIG. 7A) or from a list-based selection feature (FIG. 7B, 7C). In embodiments, a patient may access an “Appointments” module of the application, which may contain a listing of the patient's upcoming appointments. In embodiments, a patient may click on a “+” or other feature in an action bar on a page, in order to select a location for an appointment. In embodiments involving a map-based selection feature, the map may include markers which indication the location of health service centers (FIG. 7D). Optionally, if a patient taps, scrolls over, or otherwise interacts with such a marker, additional information about the location of the health service center may be provided (e.g. address, phone number, hours, etc.) (FIG. 7E). Optionally, from a marker on a map, a patient may be able to access a menu or page which permits the patient to schedule an appointment at the health service center at the corresponding marker on the map (FIG. 7F). In embodiments, a menu or page which permits a patient to schedule an appointment at a health service center allows a patient to select a particular time slot for an appointment (FIG. 7G). A patient may access different appointment times by, for example, scrolling through a listing of available times or entering a desired time and being provided with appointment times at or near the desired time. In embodiments, a patient may only have the option of selecting a day or general time of day (e.g. morning, afternoon, or evening) for an appointment, rather than a specific time slot. In embodiments, patient may be automatically scheduled for the first available appointment at the patient's selected health service center. In embodiments, a menu or page for scheduling may have tabs for one or more different time periods (e.g. morning, afternoon, and evening), in order to permit a patient to rapidly navigate time slots available at different times of day (FIG. 7G). Optionally, if a patient selects a time slot for an appointment, the patient may be presented with a screen that permits the patient review the time slot he or she has selected, and to confirm the selected appointment time (FIG. 7H). In embodiments, a patient may select a feature which permits the appointment time to be added to a computerized calendar associated with the patient (e.g. Google Calendar or Outlook calendar) (FIG. 7H). A patient may also have the option of editing a selected appointment location or time. Once a patient confirms an appointment time, the patient may receive a message which indicates that the confirmation was received (e.g. which states, for example “Thanks! We'll see you soon!”) (FIG. 7I). Optionally, such a message may disappear from the display after a set period of time. Also, once a patient has selected and confirmed an appointment time, a patient may be able to access a page in an application which permits the patient to view all of his or her upcoming appointments at health service centers associated with a health service center computer system.

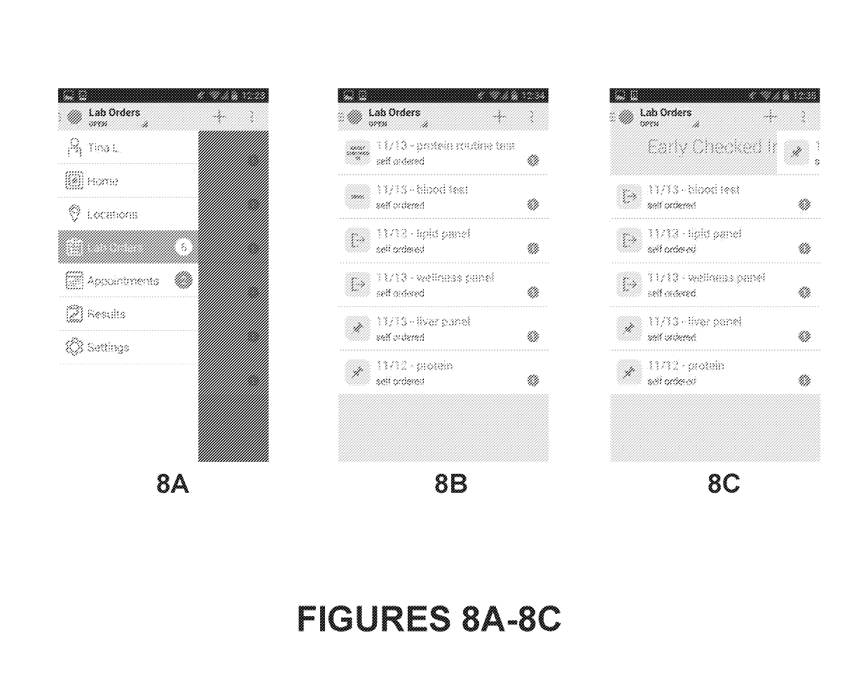

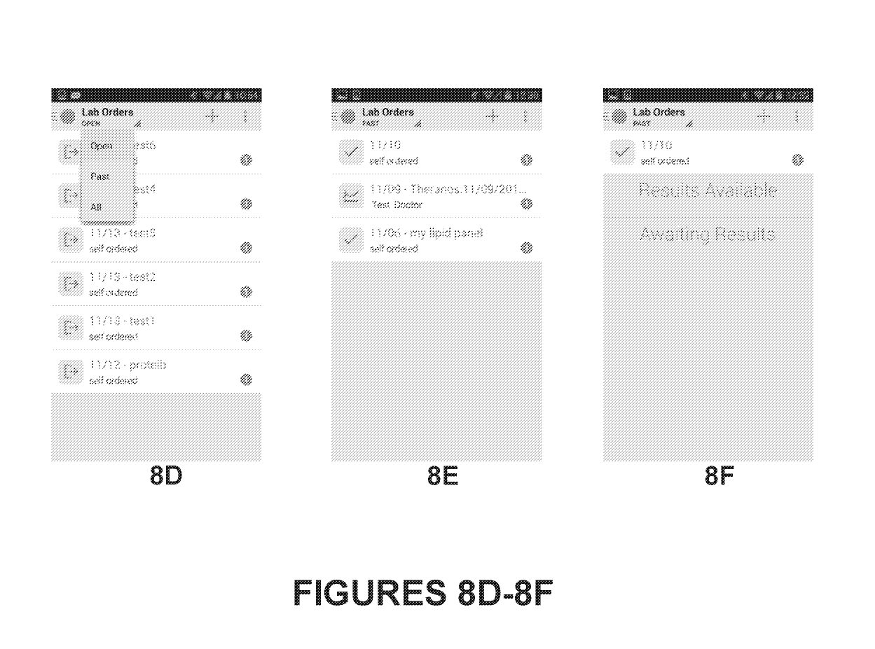

In embodiments, one or more steps of methods for appointment scheduling or check in provided herein may be performed with reference to FIG. 8. In health service center computer system application operating on a user device, a patient may have the option of reviewing or selecting various types of information related to his or her account, such as appointments that have been scheduled but not completed, appointments that have been completed, laboratory tests that have been ordered but not completed, laboratory tests that have been completed, etc. (FIG. 8A). In embodiments, if a patient performs an action relating to information on a screen (e.g. swiping the screen, typing text, or clicking a radio button), the patient may, for example, obtain more information, or trigger an action. For instance, if a patient swipes or taps an entry on the screen for laboratory orders, another screen or window may appear which contains some or all of the patient's recent or other laboratory test orders (FIG. 8B). Additional details about laboratory test orders may also be provided, such as the name of the person who ordered the laboratory test (e.g. a health care provider of the patient him or herself). Optionally, a patient may be required to perform another action relating to a specific listed laboratory order, in order to be taken to another screen or window which provides the laboratory test results. Such a screen or window may also provide other information, such as whether or not the patient has checked in for the laboratory test, details about the laboratory test, how the test is performed, or a patient's results for the test over a given period of time (FIG. 8C). In embodiments, a patient may filter his or her laboratory orders by one or more status filters, such as lab orders in open status (e.g. “early checked in” or “open”) or lab orders in a past status (e.g. “completed/awaiting results” or “results available”) (FIG. 8D, 8E). Alternatively, a patient may view all of his or her past and current laboratory orders. In embodiments, a patient may swipe, tap, or take another action relating to an item on a screen in order to obtain more information about the item, such as whether there are results available for a given laboratory test, or to view the test results (FIG. 8F).

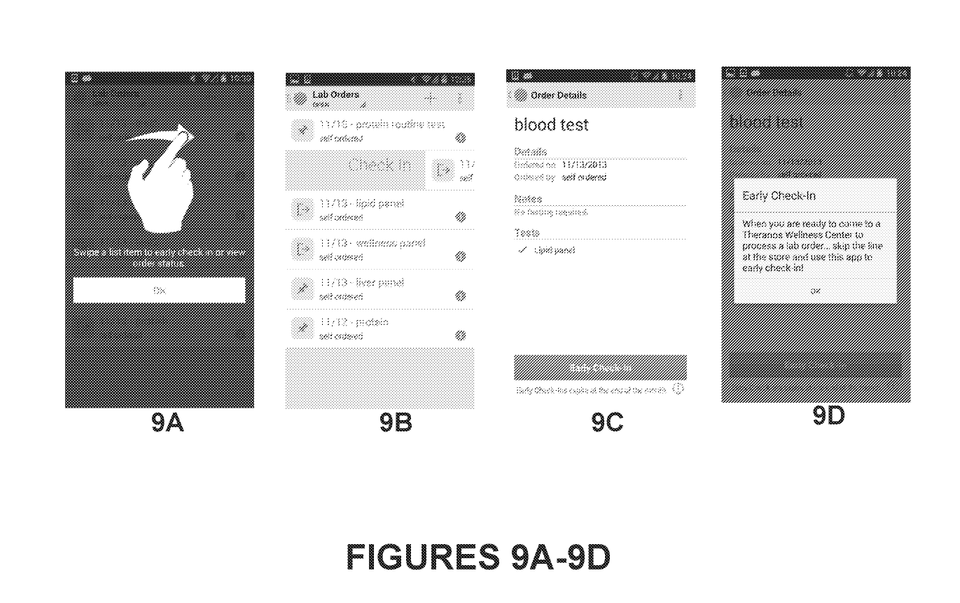

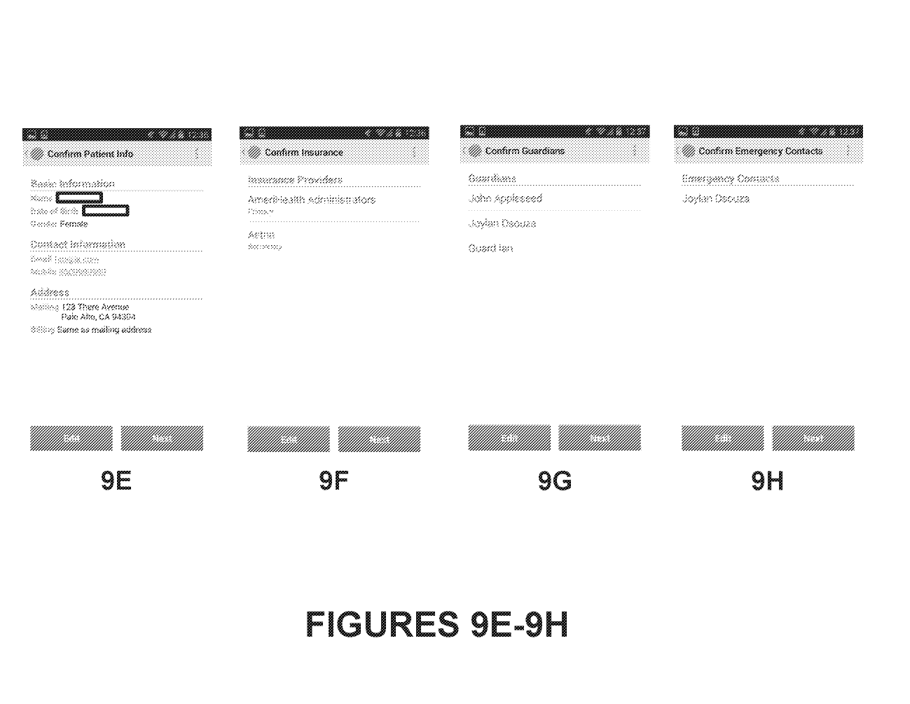

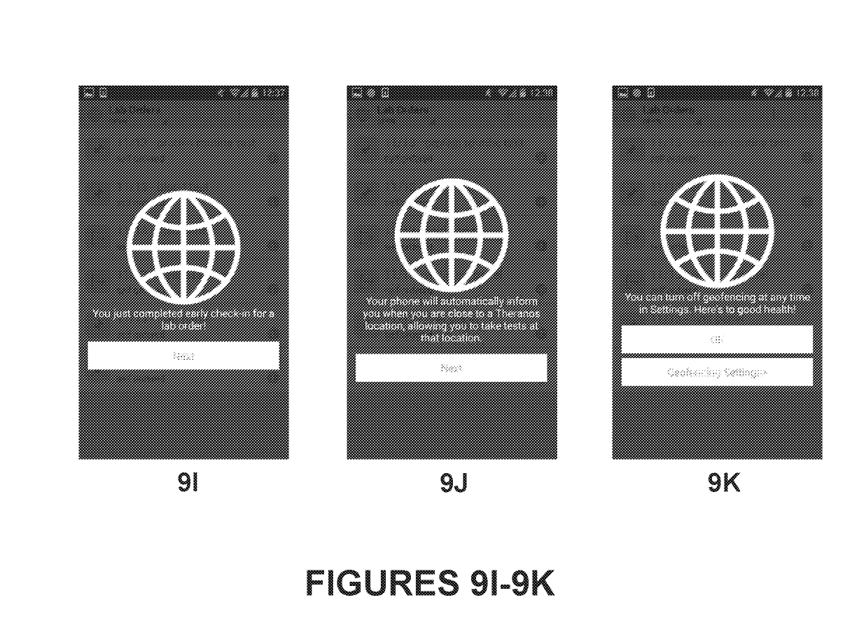

Turning now to FIG. 9, in embodiments, a patient may have the option of checking in for an appointment for a laboratory order or other procedure at a health service center from an application of a health service center computer system on a user device which lists one or more of a patient's future appointments. For example, a patient may have the option of swiping, touching, or otherwise interacting with a listing of a laboratory test, in order to check in for an appointment relating to the laboratory test (FIG. 9A). Other options, such as viewing laboratory order status may also be provided when interacting with a listing for a laboratory test. Optionally, a patient may interact with a listing of a laboratory test, and then be presented with an option to check-in for the test (FIG. 9B). In some situations, a patient may be presented with details about his or her appointment (e.g. the time, date, or tests to be performed during the appointment, a test order date, a person who ordered the test, actions for the patient to take before the appointment, etc.) when being given the option to check in for an appointment (FIG. 9C). In embodiments, a patient may be provided with the option to check in for an appointment only when the patient is at the location of a health service center, or within a certain distance of the health service center (e.g. 5 miles or less). In other embodiments, a patient may be provided with the option to check in for an appointment at a time in advance of the appointment. For instance, a patient may have the option of checking in for an appointment up to or at least 1, 2, 4, 8, 12, 16, 24, or 48 hours before a scheduled appointment (FIG. 9D). With an early-check in, a patient may have the option of providing before the time of the appointment one or more types of information which are used in association with the appointment (e.g. patient name, date of birth, insurer, address, contact information, guardians, emergency contacts, consent form for a medical or laboratory procedure, etc.), in order to reduce or eliminate information that the patient needs to provide at a health service center at the time of the appointment (FIGS. 9E, 9F, 9G, and 9H). Providing such information before the time of the appointment may, for example, reduce the waiting or procedure time for the patient at the health service center. In embodiments, a patient may have previously entered into an application some or all of the information requested as part of checking in for an appointment. In those situations, the information may be pre-loaded into a screen for the patient, and the patient may simply confirm that the information is correct. The patient may also have the option of editing of the information. Optionally, upon checking in for an appointment, a patient may receive a message indicating that the check-in procedure is complete (FIG. 9I). In embodiments, a once a patient has checked in for an appointment ahead of the scheduled appointment time and at a location remote from the health service center where the appointment is schedule, the patient may have the option of being notified by an application from the health service center computer system when he or she is in the vicinity of the health service center (e.g. a “geo-fencing” capability). Optionally, the patient may be informed of this feature when he or she checks in for an appointment at a location remote from the health service center (FIG. 9J). In embodiments, the patient may have the option of turning on or off this feature at the time of check in or other times (FIG. 9K).

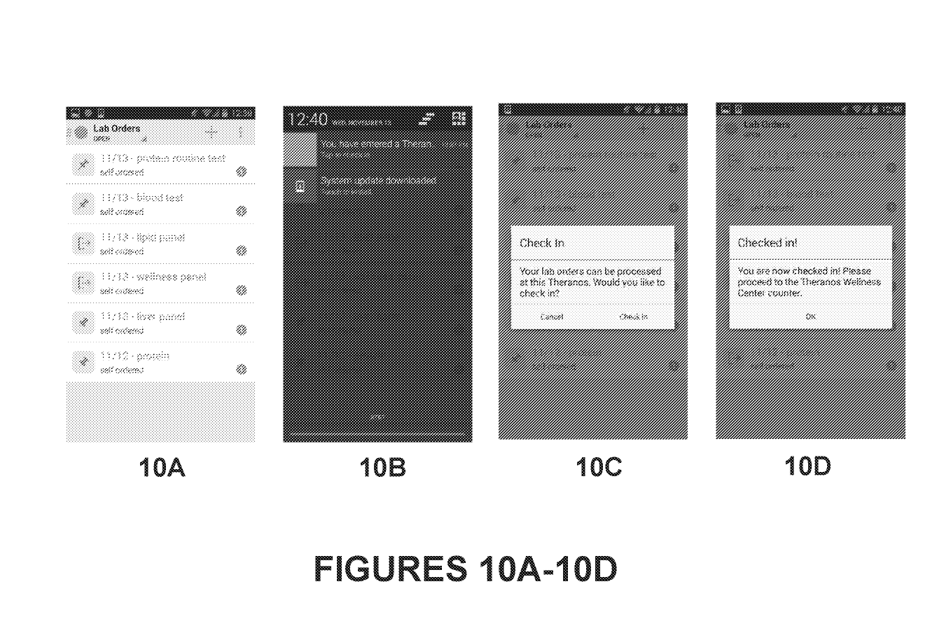

Turning now to FIG. 10, in embodiments, a patient may have a user device operating an application of a health service center computer system which can receive information regarding the location of the patient, and provide relevant information to the patient based on the patient's location. Optionally, if a patient is within a certain distance of a health service center (e.g. inside a health service center or no more than 10 feet from the entrance of a health service center, etc.), a patient may receive a notification indicating that he or she is inside or close to a health service center (FIG. 10A). The notification may take any appropriate form, such as a visual or audio cue. For example, a visual cue may be a symbol in a display of a user device of an application of a health service center computer system, such as a particular shape or combination of letters (FIG. 10A, 10B). After or simultaneous with a notification that a patient is inside or close to health service center, a patient may also be given the option of performing one or more steps relating to his or her appointment, such as checking in for an appointment (FIG. 10B). When checking in for an appointment, a patient may be given an option to confirm that he or she would like to check in for the appointment (FIG. 10C). Upon check in at the health service center, a patient may be given instructions about one or more next steps to take, such as where to go or what to do to prepare for the appointment (FIG. 10D).

In embodiments, any of the actions described herein which may be performed by a patient may be performed by the patient on a user device (e.g. a patient's smart phone or personal computer). The user device may be operatively connected to a health service center computer system and may run an app from the health service center computer system. In embodiments, a health service center computer system may contain hardware, software, or a combination thereof for performing methods provided herein, or portions thereof.

While various embodiments of the invention are shown and described herein, these are provided by way of example only. It should be understood that there is no intent to limit the invention to the particular forms disclosed, but rather, the invention is to cover all modifications, equivalents, and alternatives falling within the spirit and scope of the invention provided herein. For example, although the foregoing systems and methods are generally described in the context of appointments at health service centers, in embodiments, the systems and methods provided herein may be used with appointment scheduling and check-in for other types of services and appointment systems (e.g. at restaurants, salons, amusement centers, etc.).

Description and disclosure of examples of reagents, assays, methods, kits, devices, and systems which may use, or be used with, method, devices, and systems disclosed herein may be found, for example, in U.S. Pat. No. 8,088,593; U.S. Pat. No. 8,380,541; U.S. patent application Ser. No. 13/769,798, filed Feb. 18, 2013; U.S. patent application Ser. No. 13/769,779, filed Feb. 18, 2013; U.S. patent application Ser. No. 13/769,817, filed Feb. 18, 2013; U.S. patent application Ser. No. 13/769,818, filed Feb. 18, 2013; U.S. patent application Ser. No. 13/769,820, filed Feb. 18, 2013; U.S. patent application Ser. No. 13/244,947 filed Sep. 26, 2011; PCT/US2012/57155, filed Sep. 25, 2012; U.S. application Ser. No. 13/244,946, filed Sep. 26, 2011; U.S. patent application Ser. No. 13/244,949, filed Sep. 26, 2011; and U.S. Application Ser. No. 61/673,245, filed Sep. 26, 2011, the disclosures of which patents and patent applications are all hereby incorporated by reference in their entireties.

This application claims the benefit of, and priority to, each of U.S. Provisional Patent Application No. 61/875,108, filed Sep. 8, 2013; U.S. Provisional Patent Application No. 61/899,869, filed Nov. 4, 2013; U.S. Provisional Patent Application No. 61/900,985, filed Nov. 6, 2013, and U.S. Provisional Patent Application No. 62/001,542, filed May 21, 2014, the content of each of which is hereby incorporated by reference in its entirety for all purposes.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. Any feature, whether preferred or not, may be combined with any other feature, whether preferred or not. It should also be understood that while the invention provided herein has been described herein using a limited number of terms and phrases for purposes of expediency, the invention could also be described using other terms and phrases not provided herein which also accurately describe the invention. The appended claims are not to be interpreted as including means-plus-function limitations, unless such a limitation is explicitly recited in a given claim using the phrase “means for.” It should be understood that as used in the description herein and throughout the claims that follow, the meaning of “a,” “an,” and “the” includes plural reference unless the context clearly dictates otherwise. For example, a reference to “an assay” may refer to a single assay or multiple assays. Also, as used in the description herein and throughout the claims that follow, the meaning of “in” includes “in” and “on” unless the context clearly dictates otherwise. As used in the description herein and through the claims that follow, a first object described as containing “at least a portion” of a second object may contain the full amount of/the complete second object. As used in the description herein and throughout the claims that follow, the terms “comprise”, “include”, and “contain” and related tenses are inclusive and open-ended, and do not exclude additional, unrecited elements or method steps. Also, the presence of broadening words and phrases such as “one or more,” “at least,” “but not limited to” or other like phrases in some instances shall not be read to mean that the narrower case is intended or required in instances where such broadening phrases may be absent. Finally, as used in the description herein and throughout the claims that follow, the meaning of “or” includes both the conjunctive and disjunctive unless the context expressly dictates otherwise. Thus, the term “or” includes “and/or” unless the context expressly dictates otherwise.

This document contains material subject to copyright protection. The copyright owner (Applicant herein) has no objection to facsimile reproduction by anyone of the patent documents or the patent disclosure, as they appear in the US Patent and Trademark Office patent file or records, but otherwise reserves all copyright rights whatsoever. The following notice shall apply: Copyright 2013-14 Theranos, Inc.

1-34. (canceled)

35. A health service center computer system, wherein the health service center computer system comprises a processor which is programmed to perform at least one of a method selected from the group of methods consisting of:
a) a method for evaluating the risk of a patient's tardy arrival for a scheduled appointment at a time at a health service center, the method comprising:
obtaining at least a first tardiness risk factor and a second tardiness risk factor for the patient for the scheduled appointment;
obtaining, from a computer memory device and with the aid of a processor, a first quantified tardiness risk value for the first tardiness risk factor and a second quantified tardiness risk value for the second tardiness risk factor; and
combining, according to an algorithm and with the aid of a processor, the first quantified tardiness risk value and the second quantified tardiness risk value, to generate a total tardiness risk value;
b) a method of managing a patient's arrival at a health service center, the method comprising receiving by a health service center computer system information indicating that a patient has brought an NFC-enabled device into close proximity with an NFC tag in a display in the health service center; and
c) a method of managing a patient's arrival at a health service center, the method comprising receiving by a health service center computer system information indicating that a computing device maintained by the patient is within a selected distance of the health service center.

36. A system for managing appointments, comprising:
a health service center computer system, wherein the health service center computer system comprises a processor which is programmed to perform the method of ***;
a user device, wherein the is operatively connected to the health service center computer system.

37. The system of claim 36, wherein the user device is a health service center terminal, wherein the health service center terminal is located in a health service center.

38. The system of claim 37, wherein the health service center terminal comprises a display.

39. The system of claim 38, wherein the health service center terminal further comprises a keyboard.

40. The system of claim 36, wherein the health service center computer system and the user device are operatively connected via a local network.

41. The system of claim 36, wherein the health service center computer system and the user device are operatively connected via the Internet.

42. The system of claim 36, wherein the system further comprises a display containing a NFC tag, wherein the display is located in the health service center.

43. A non-transitory computer-readable medium comprising machine-executable code for implementing a method for evaluating the risk of a patient's tardy arrival for a scheduled appointment at a time at a health service center, the method comprising a method selected from the group of methods consisting of:
a) a method for evaluating the risk of a patient's tardy arrival for a scheduled appointment at a time at a health service center, the method comprising:
obtaining at least a first tardiness risk factor and a second tardiness risk factor for the patient for the scheduled appointment;
obtaining, from a computer memory device and with the aid of a processor, a first quantified tardiness risk value for the first tardiness risk factor and a second quantified tardiness risk value for the second tardiness risk factor; and
combining, according to an algorithm and with the aid of a processor, the first quantified tardiness risk value and the second quantified tardiness risk value, to generate a total tardiness risk value;
b) a method of managing a patient's arrival at a health service center, the method comprising receiving by a health service center computer system information indicating that a patient has brought an NFC-enabled device into close proximity with an NFC tag in a display in the health service center; and
c) a method of managing a patient's arrival at a health service center, the method comprising receiving by a health service center computer system information indicating that a computing device maintained by the patient is within a selected distance of the health service center.

44-45. (canceled)

46. The health service center computer system of claim 35, wherein said processor is programmed to perform method a) comprising a method for evaluating the risk of a patient's tardy arrival for a scheduled appointment at a time at a health service center, wherein said method a) further comprises comparing the total tardiness risk value to a threshold tardiness risk value, and, if the total tardiness risk value exceeds the threshold tardiness risk value, taking an action.

47. The health service center computer system of claim 46, wherein the action is selected from the group consisting of: contacting the patient, assigning the patient a new scheduled appointment time, and assigning a different patient an appointment at the patient's original scheduled appointment time.

48. The health service center computer system of claim 35, wherein said processor is programmed to perform method a) comprising a method for evaluating the risk of a patient's tardy arrival for a scheduled appointment at a time at a health service center, wherein said method a) further comprises, based on the value of the total tardiness risk, taking an action, wherein the action is selected from the group consisting of: contacting the patient, assigning the patient a new scheduled appointment time, and assigning a different patient an appointment at the patient's original scheduled appointment time.

49. The health service center computer system of claim 35, wherein said processor is programmed to perform method b) a method of managing a patient's arrival at a health service center, wherein the information comprises the time of day that the patient brought the NFC-enabled device into close proximity with the NFC tag in the display at the health service center.

50. The health service center computer system of claim 49, wherein said processor is programmed to perform method b) a method of managing a patient's arrival at a health service center, further comprising comparing by a processor in the health service center computer system: i) a scheduled appointment time for the patient to ii) the time of day that the patient brought the NFC-enabled device into close proximity with the NFC tag in the display at the health service center.

51. The health service center computer system of claim 35, wherein said processor is programmed to perform method c) a method of managing a patient's arrival at a health service center, wherein the patient has a scheduled appointment at a time at the health service center, and wherein the selected distance is no greater than 1 mile.
